(19) United States
(12) Patent Application Publication
(10) Pub. No.: US 2014/0301935 A1
(43) Pub. Date: Oct. 9, 2014

(54) OLIGOPHENYLENE MONOMERS AND POLYMERIC PRECURSORS FOR PRODUCING GRAPHENE NANORIBBONS

(71) Applicants: BASF SE, Ludwigshafen (DE); Max-Planck-Gesellschaft zur Foerderung der Wissenschaften e.V., Muenchen (DE)

(72) Inventors: Sorin Ivanovici, Heidelberg (DE); Matthias Georg Schwab, Mannheim (DE); Xinliang Feng, Mainz (DE); Klaus Muellen, Koeln (DE)

(73) Assignees: BASF SE, Ludwigshafen (DE); Max-Planck-Gesellschaft zur Foerderung der Wissenschaften e.V., Muenchen (DE)

(21) Appl. No.: 14/354,329

(22) PCT Filed: Oct. 24, 2012
(86) PCT No.: PCT/IB2012/055843

§ 371 Date: Apr. 25, 2014

Related U.S. Application Data
(60) Provisional application No. 61/551,458, filed on Oct. 26, 2011.

(51) Int. Cl. C01B 31/04; C08G 61/10; C07C 17/30; C07C 25/18

(52) U.S. Cl. CPC C01B 31/0446; C07C 25/18; C08G 61/10; C07C 17/30; C01B 2204/06; C08G 2261/312; C08G 2261/412; C08G 2261/148
USPC 423/448; 570/182; 528/397; 528/8; 570/201

(57) ABSTRACT

Oligophenylene monomers for the synthesis of polymeric precursors for the preparation of graphene nanoribbons, the polymeric precursors, and methods for preparing them, as well as methods for preparing the graphene nanoribbons from the polymeric precursors and the monomers are provided.

The present invention concerns oligophenylene monomers for the synthesis of polymeric precursors for the preparation of graphene nanoribbons, the polymeric precursors, and methods for preparing them, as well as methods for preparing the graphene nanoribbons from the polymeric precursors and the monomers.

Graphene, an atomically thin layer from graphite, has received considerable interest in physics, material science and chemistry since the recent discovery of its appealing electronic properties. These involve superior charge carrier mobility and the quantum Hall effect. Moreover, its chemical robustness and material strength make graphene an ideal candidate for applications ranging from transparent conductive electrodes to devices for charge and energy storage.

Graphene nanoribbons (GNRs) are linear structures that are derived from the parent graphene lattice. Their characteristic feature is high shape-anisotropy due to the increased ratio of length over width. Currently, their usage in yet smaller, flatter and faster carbon-based devices and integrated circuits is being widely discussed in material science. In contrast to graphene, armchair-type GNRs exhibit a band gap that can be adjusted by their width. Their length becomes relevant when GNRs are to be used in devices such as field-effect transistors (FETs) for which a minimum channel width has to be bridged. The same holds for the potential replacement of copper or gold in nanoscale conducting pathways. At the same time the edge structure of the GNRs will have a strong impact. Computational simulations and experimental results on smaller nanographenes suggest that GNRs exhibiting nonbonding π-electron states at zigzag edges could be used as active component in spintronic devices.

The reason why there are so few chemically defined GNRs is the considerable complexity that governs design, chemical preparation and processing of these structures. In the recent past, only few synthetic attempts have been published addressing the fabrication of GNRs of defined geometry, width, length, edge structure and heteroatom-content. Based on the reaction environment the studies on the synthetic bottom-up fabrication of GNRs can be further divided into solution- and surface-based routes.

For solution-based approaches using oligophenylene precursors a polymer is typically prepared in a first step which is subsequently converted into the graphitic structure by Scholl-type oxidative cyclodehydrogenation. However, the design of the parent monomer must be carefully adjusted in order to guarantee for a suitable arrangement of the aromatic units upon the chemistry-assisted graphitization into the final GNR structure.

J. Wu, L. Gherghel, D. Watson, J. Li, Z. Wang, C. D. Simpson, U. Kolb, and K. Müllen, Macromolecules 2003, 36, 7082-7089 report the synthesis of graphitic nanoribbons obtained by intramolecular oxidative cyclodehydrogenation of soluble branched polyphenylenes, which were prepared by repetitive Diels-Alder cycloaddition of 1,4-bis(2,4,5-triphenylcyclopentadienone-3-yl)benzene and diethynylterphenyl. The obtained graphene ribbons are not linear but rather contain statistically distributed “kinks” due to the structural design of the polyphenylene precursor.

X. Yang., X. Dou, A. Rouhanipour, L. Zhi, H. J. Räder, and K. Müllen, JACS Communications, Published on Web Mar. 7, 2008, report the synthesis of two-dimensional graphene nanoribbons. Suzuki-Miyaura coupling of 1,4-diiodo-2,3,5,6-tetraphenylbenzene with 4-bromophenylboronic acid gives dibromo-hexaphenylbenzene, which is converted into the bis-boronic ester. Suzuki-Miyaura polymerization of the bis-boronic ester with diiodobenzene furnished polyphenylenes in a strongly sterically hindered reaction. Intramolecular Scholl reaction of the polyphenylene with FeCl₃ as oxidative reagent provides graphene nanoribbons.

Y. Fogel, L. Zhi, A. Rouhanipour, D. Andrienko, H. J. Räder, and K. Müllen, Macromolecules 2009, 42, 6878-6884 report the synthesis of a homologous series of five monodisperse ribbon-type polyphenylenes, with rigid divenzopyrene cores in the repeat units, by microwave-assisted Diels-Alder reaction. The size of the obtained polyphenylene ribbons ranges from 132 to 372 carbon atoms in the aromatic backbone which incorporates up to six dibenzopyrene units. Because of the flexibility of the backbone and the peripheral substitution with dodecyl chains, the polyphenylene ribbons are soluble in organic solvents. In a further reaction step, ribbon-type polycyclic aromatic hydrocarbons (PAHs) are prepared by cyclodehydrogenation.

All three methods suffer from drawbacks regarding the final graphene nanoribbon.

In the first case, the resulting graphene nanoribbons are ill-defined due to the statistically arranged “kinks” in their backbone. Furthermore the molecular weight is limited due to the sensitivity of the A2B2-type polymerization approach to abberations from stochiometry. No lateral solubilizing alkyl chains have been introduced into the graphene nanoribbons.

The second case suffers also from the stochiometry issue due to the underlying A2B2-stochiometry of the A2B2-type Suzuki protocol and the sterical hindrance of 1,4-diiodo-2,3,5,6-tetraphenylbenzene.

The third case makes use of a step-wise synthesis which provides very defined cutouts from graphene nanoribbons but is impracticable for the fabrication of high-molecular weight species.

It is an object of the present invention to provide new methods for the production of graphene nanoribbons. It is a further object of the present invention to provide suitable polymeric precursors for the preparation of graphene nanoribbons, as well as methods and suitable monomeric compounds for preparing such polymeric precursors.

The problem is solved by oligophenylene monomers of general formulae A, B, C, D, E and F for the synthesis of polymeric precursors for the preparation of graphene nanoribbons of general formulae A, B, C, D, E and F

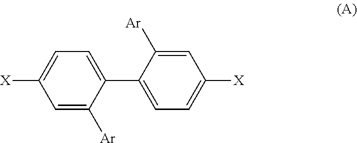

wherein
Ar is selected from

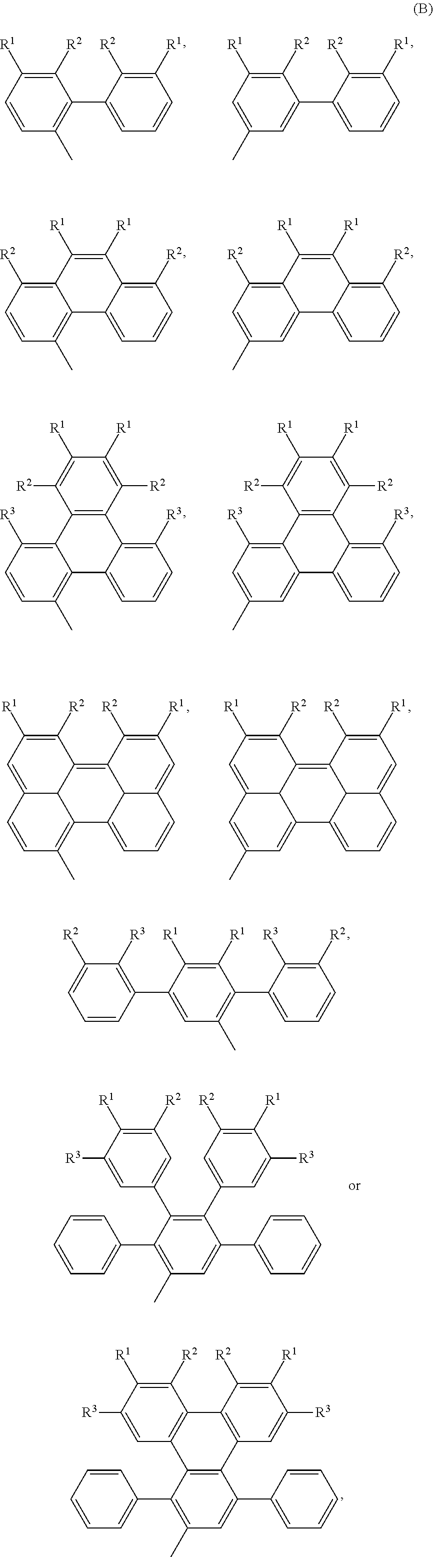

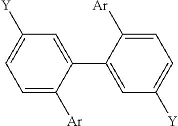

wherein
Ar is selected from

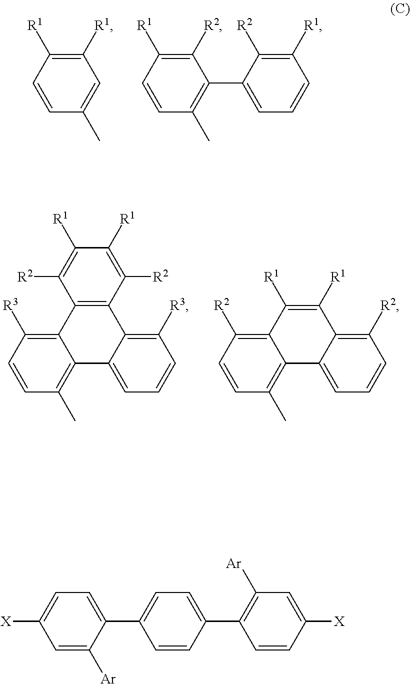

wherein
Ar is selected from

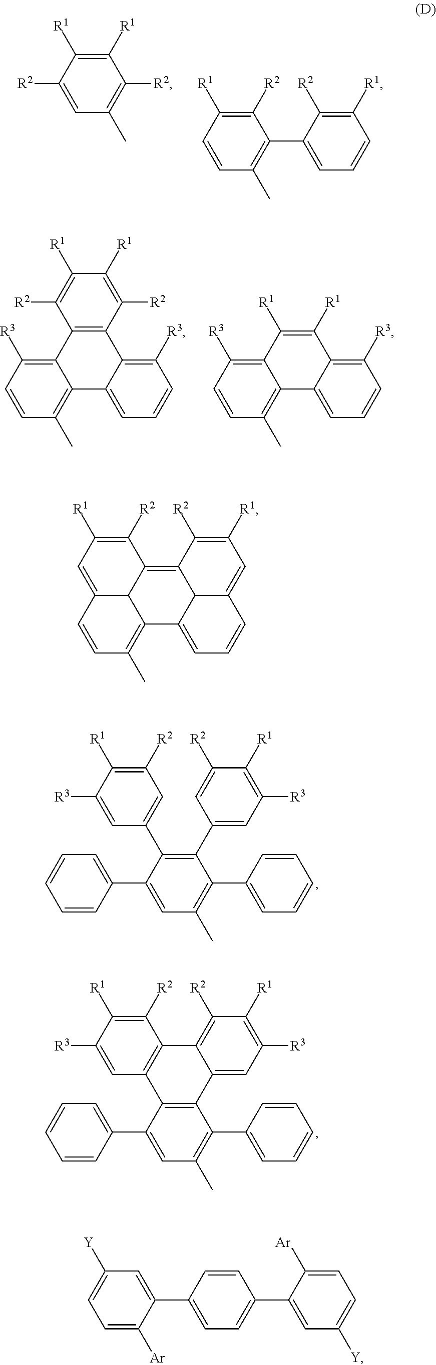

wherein
Ar is

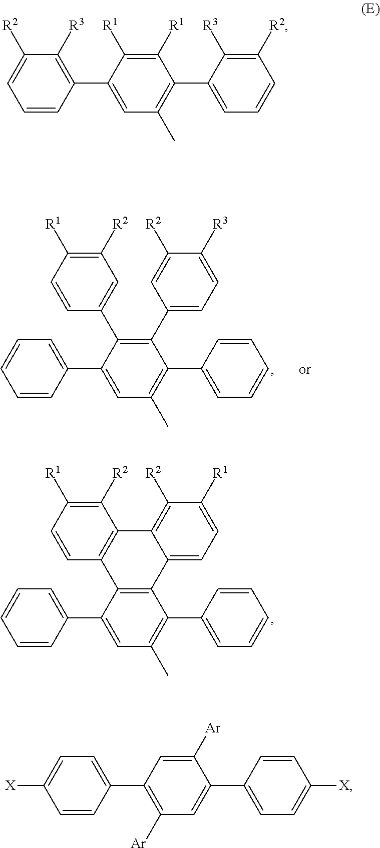

wherein
Ar is

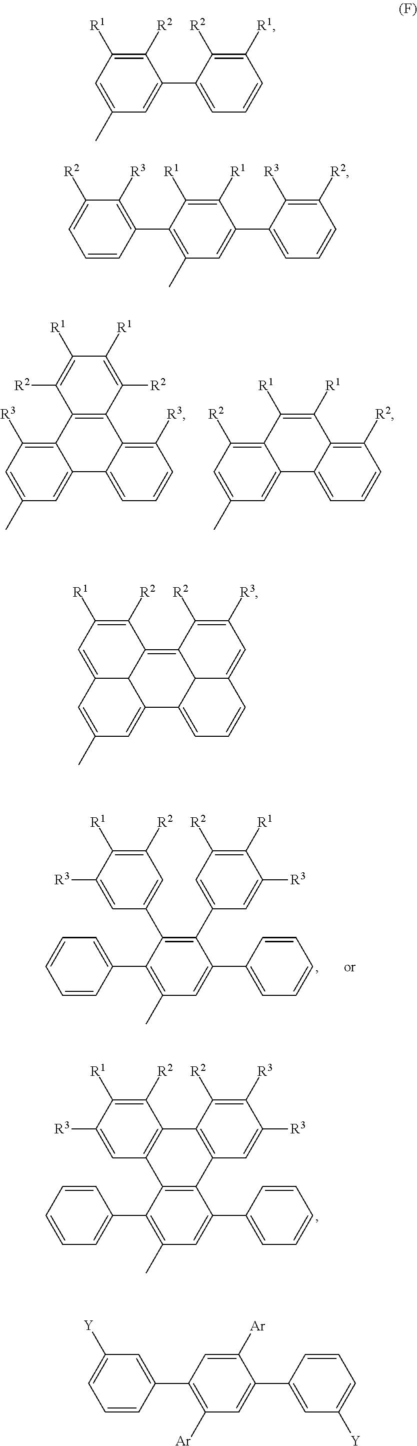

wherein
Ar is

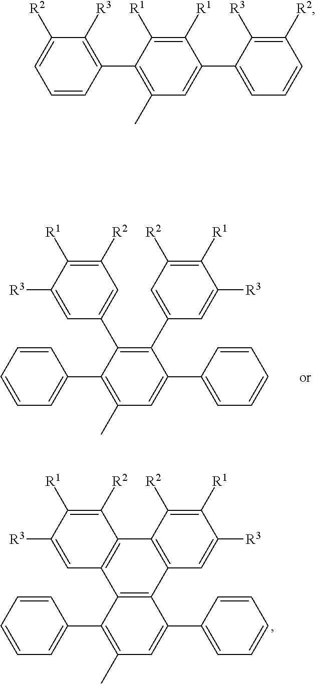

wherein, in each of formulae A, B, C, D, E and F,

X, Y is halogene, trifluoromethylsulfonate or diazonium,

R¹, R², R³ are independently of each other H, halogene, —OH, —NH₂, —CN, —NO₂ a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue.

In some preferred embodiments, R² and R³ are hydrogen.

Preferred oligophenylene monomers are those of formulae I, II, III and IV:

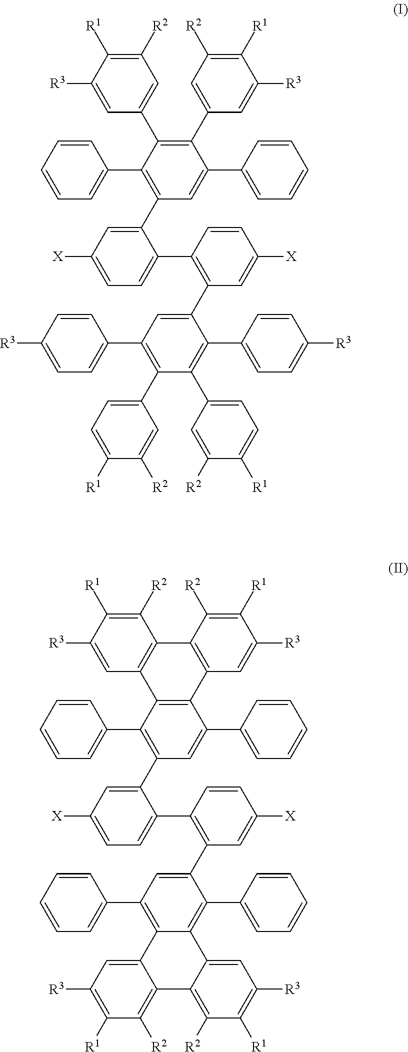

wherein
R¹, R², R³═H, halogene, —OH, —NH₂, —CN, —NO₂, a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue,
X=halogene, trifluoromethylsulfonate or diazonium.

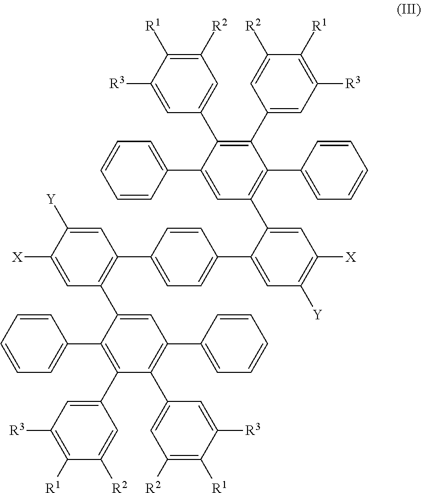

wherein
R¹, R², R³=H, halogene, —OH, —NH₂, —CN, —NO₂, a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue, and
X=halogene and Y═H (IIIa) or X═H and Y=halogene (IIIb)

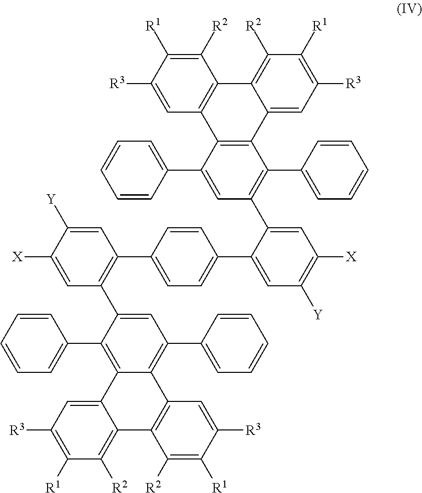

wherein
R¹, R², R³═H, halogene (F, Cl, Br, I—OH), —NH₂, —CN, —NO₂, a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue, and
X=halogene and Y═H (IVa) or X═H and Y=halogene (IVb).
with the proviso that R³═H if X═H and Y=halogene.

Preferably, R¹, R² and R³ are independently of each other hydrogen, C₁-C₃₀ alkyl, C₁-C₃₀ alkoxy, C₁-C₃₀ alkylthio, C₂-C₃₀ alkenyl, C₂-C₃₀ alkynyl, C₁-C₃₀ haloalkyl, C₂-C₃₀ haloalkenyl and haloalkynyl, e.g. C₁-C₃₀ perfluoroalkyl.

C₁-C₃₀ alkyl can be linear or branched, where possible.

Examples are methyl, ethyl, n-propyl, isopropyl, n-butyl, sec.-butyl, isobutyl, tert.-butyl, n-pentyl, 2-pentyl, 3-pentyl, 2,2-dimethylpropyl, 1,1,3,3-tetramethylpentyl, n-hexyl, 1-methylhexyl, 1,1,3,3,5,5-hexamethylhexyl, n-heptyl, isoheptyl, 1,1,3,3-tetramethylbutyl, 1-methylheptyl, 3-methylheptyl, n-octyl, 1,1,3,3-tetramethylbutyl and 2-ethylhexyl, n-nonyl, decyl, undecyl, dodecyl, tridecyl, tetradecyl, pentadecyl, hexadecyl, heptadecyl, octadecyl, eicosyl, heneicosyl, docosyl, tetracosyl or pentacosyl.

C₁-C₃₀ alkoxy groups are straight-chain or branched alkoxy groups, e.g. methoxy, ethoxy, n-propoxy, isopropoxy, n-butoxy, sec-butoxy, tert-butoxy, amyloxy, isoamyloxy or tert-amyloxy, heptyloxy, octyloxy, isooctyloxy, nonyloxy, decyloxy, undecyloxy, dodecyloxy, tetradecyloxy, pentadecyloxy, hexadecyloxy, heptadecyloxy and octadecyloxy.

The term “alkylthio group” means the same groups as the alkoxy groups, except that the oxygen atom of the ether linkage is replaced by a sulfur atom.

C₂-C₃₀ alkenyl groups are straight-chain or branched alkenyl groups, such as e.g. vinyl, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

C₂₋₃₀ alkynyl is straight-chain or branched such as, for example, ethynyl, 1-propyn-3-yl, 1-butyn-4-yl, 1-pentyn-5-yl, 2-methyl-3-butyn-2-yl, 1,4-pentadiyn-3-yl, 1,3-pentadiyn-5-yl, 1-hexyn-6-yl, cis-3-methyl-2-penten-4-yn-11-yl, trans-3-methyl-2-penten-4-yn-1-yl, 1,3-hexadiyn-5-yl, 1-octyn-8-yl, 1-nonyn-9-yl, 1-decyn-10-yl, or 1-tetracosyn-24-yl.

C₁-C₃₀-perfluoroalkyl is a branched or unbranched radical such as for example —CF₃, —CF₂CF₃, —CF₂CF₂CF₃, —CF(CF₃)₂, —(CF₂)₃CF₃ or —C(CF₃)₃.

The terms “haloalkyl, haloalkenyl and haloalkynyl” mean groups given by partially or wholly substituting the abovementioned alkyl group, alkenyl group and alkynyl group with halogen.

Aryl is usually C₆-C₃₀ aryl, which optionally can be substituted, such as, for example, phenyl, 4-methylphenyl, 4-methoxyphenyl, naphthyl, biphenylyl, terphenylyl, pyrenyl, fluorenyl, phenanthryl, anthryl, tetracyl, pentacyl and exacyl.

Preferably, R² and R³ are hydrogen.

Preferably, X and Y are Cl or Br.

The problem is further solved by polymeric precursors for the preparation of graphene nanoribbons having repeating units of general formulae V, VI, VII, VIII, IX and X.

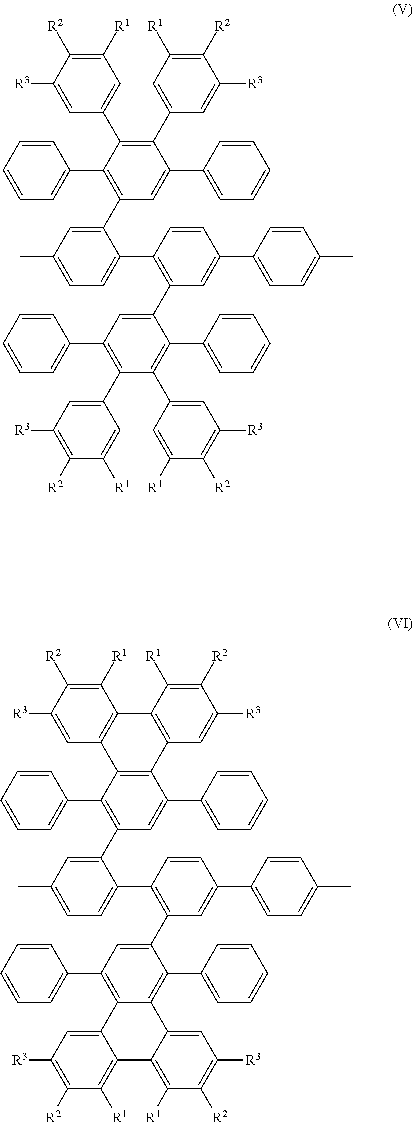

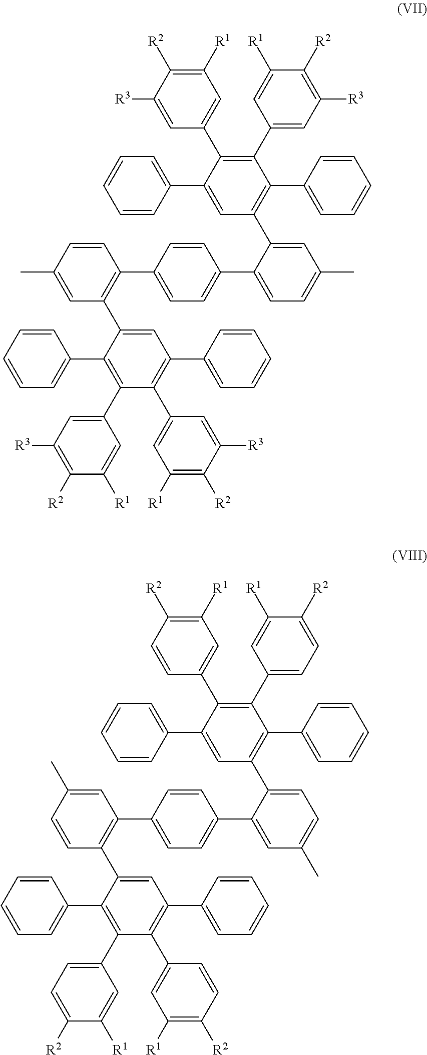

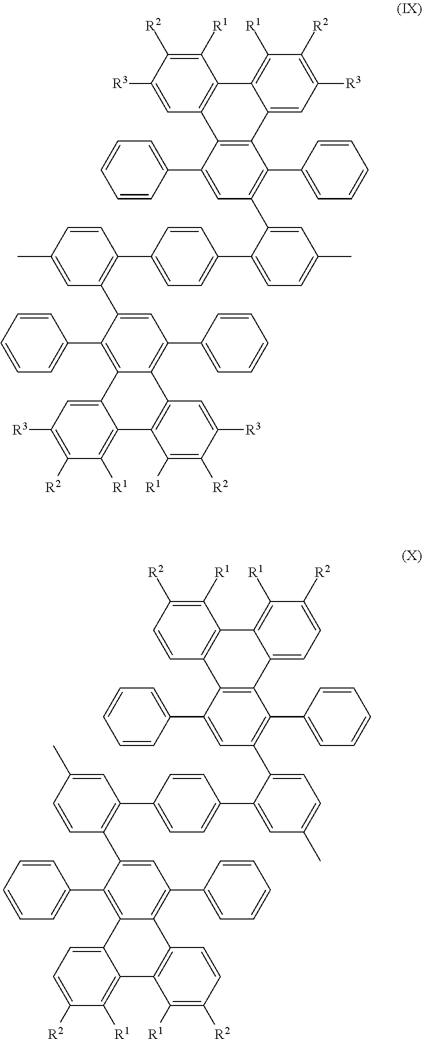

wherein
R¹, R², R³ are independently of each other H, halogene, —OH, —NH₂, —CN, —NO₂, a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or are an optionally substituted aryl, alkylaryl or alkoxyaryl residue.

Preferably, R² and R³ in formulae V-X are hydrogen.

In formulae I-X, X is preferably Cl or Br, and R¹ is preferably H or a linear or branched C₈-C₂₆ alkyl, in particular H or a linear or branched C₁₀-C₂₄ alkyl.

In one embodiment, an oligophenylene monomer of general formula I or II is used for the preparation of the polymeric precursor by reacting it with an paraphenylenediboronic acid or -diboronic acid ester via a Suzuki-Miyaura polycondensation.

The Suzuki-Miyaura reaction represents a well-established cross-coupling protocol which has been used for the build-up of functional molecules and polymers. The robust palladium(0)-mediated catalytic cycle is particularly useful for carbon-carbon bond formation between aromatic halides and arylboronic acids or their corresponding esters.

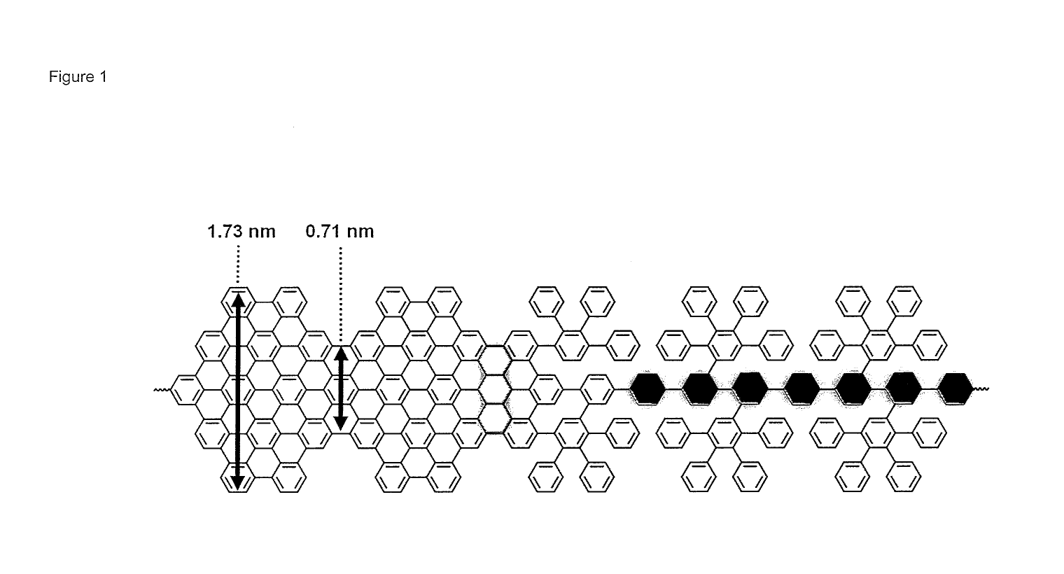

When applied as a polycondensation reaction a pair of complementarily functionalized monomers needs to be chosen. For the synthesis of GNRs via a Suzuki-Miyaura polycondensation the structural design is illustrated in FIG. 1.

The polymer can be rationalized as a laterally extended poly(para-phenylene) whose backbone chain is composed of 1,4-connected benzene rings that originate from the oligophenylene monomer and the diboronic acid.

The overlap between the repeat units of the final nanoribbons is achieved through three fused benzene units. The GNRs possess an armchair-type edge which follows the overall saw blade periphery of the graphitic structure. The maximum diameter as derived from computational analysis is 1.73 nm and narrows down to 0.71 nm at the neck position (MMFF94s). These dimensions are significantly larger than in the case of the literature-known GNRs prepared from synthetic bottom-up approaches.

For the synthesis of a suitable polymer precursor for the preparation of Suzuki-based GNRs two halogen functions are introduced on a oligophenylene unit. Polycondensation with a 1,4-functionalized diboronic acid followed by cyclodehydrogenation then leads to the formation of the target structure depicted in FIG. 1.

The oligophenylene monomer I can be synthesized as summarized below in Schemes 1 to 3.

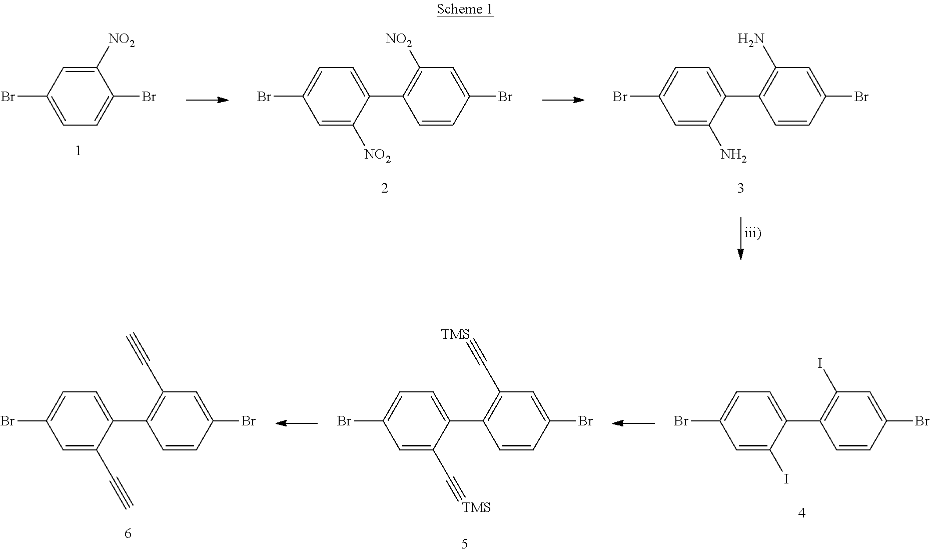

In a first reaction sequence, the intermediate 4,4′-dibromo-2,2′-diethynyl-1,1′-biphenyl 6 can be synthesized via a five-step route from commercially available 1,4-dibromo-2-nitrobenzene 1 (Scheme 1). Ullmann-type homocoupling of 1 can be used for the build-up of the biphenyl backbone. The reaction can be achieved in the melt at 190° C. in the presence of copper powder. Due to the activating effect of the electron-withdrawing nitro groups of 1, the coupling only proceeded at the bromine atoms in the desired 1-position. The next step consists in the reduction of the nitro groups to yield the functionalized biphenyl 3. This step can be realized by hydrogenation of 4,4′-dibromo-2,2′-dinitro-1,1′-biphenyl 2 using tin powder under acidic conditions.

Diamine 3 can be directly used for the next step without further purification. Diazotation under Sandmeyer conditions followed by treatment with potassium iodide successfully leads to the synthesis of unreported 4,4′-dibromo-2,2′-diiodo-1,1′-biphenyl 4. However, the mono-iodinated by-product is also observed accounting for a moderate yield in this step. Separation of both products can be achieved by column chromatography. In the next step, Sonogashira-Hagihara cross-coupling of 4 with trimethylsilyl acetylene in the presence of bis(triphenylphosphine)-palladiumchloride(II) and copper(II) iodide yields the protected bisacetylene 5.

Using potassium carbonate as base finally results in the formation of 4,4′-dibromo-2,2′-diethynyl-1,1′-biphenyl 6 at room temperature. The reaction works well when a 1/1 mixture of THF and methanol is used.

Diels-Alder [4+2] cycloaddition of acetylenes to tetraphenylcyclopentadienones is known to be a versatile method for the synthesis of large oligophenylene precursors. By this reaction, the size of the molecule is significantly increased in one single synthetic step which is in general high-yielding. The tetraphenylcyclopentadienones 11 can be prepared according to literature-known procedures. Scheme 2 illustrates the synthetic route to the 1,2-bis(4 alkylphenyl)ethane-1,2-diones 9 which can be typically used for the build-up of the tetraphenylcyclopentadienone backbone. In principle, they can be decorated with any desired alkyl chain that will confer solubility to the final nanographene molecules. Suitable examples are branched 3,7-dimethyloctyl and linear dodecyl chains. Knoevenagel condensation with diphenylacetone 10 is then used to prepare the bisalkyl tetraphenylcyclopentadienones 11 according to Scheme 3.

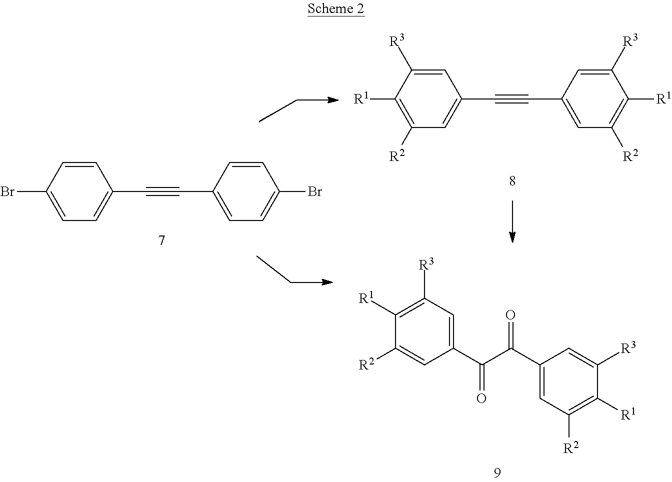

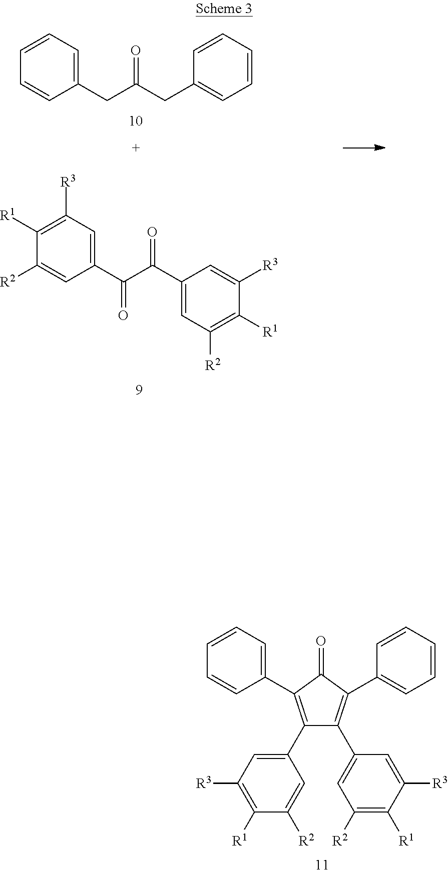

With 4,4′-dibromo-2,2′-diethynyl-1,1′-biphenyl 6 and the tetraphenylcyclopentadienones 11 at hand, the preparation of the oligophenylene monomer for the synthesis of the laterally extended poly(para-phenylenes) via Suzuki polycondensation is accessible.

Diels-Alder reaction of 6 and 11 in ortho-xylene at 160° C. using 300 W microwave irradiation yields the dendronized biphenyl 13 according to Scheme 4.

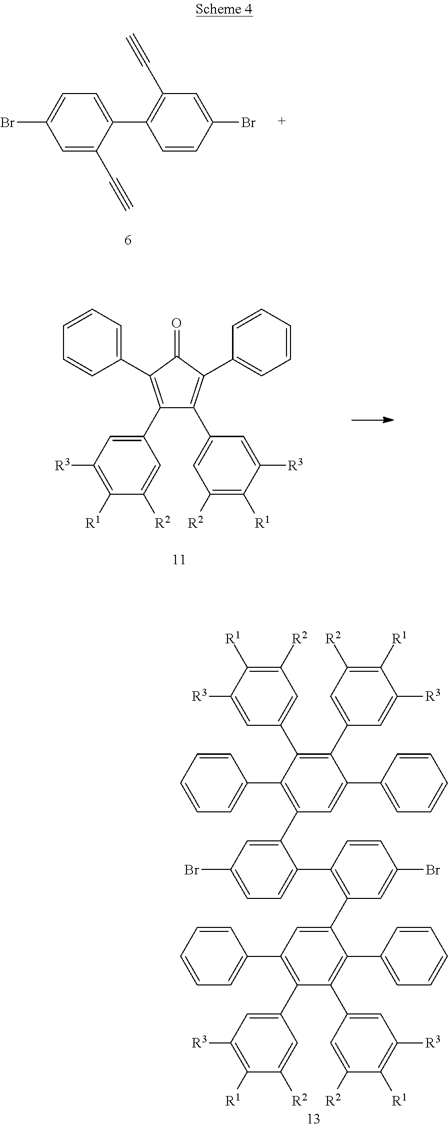

For the following A₂B₂-type polycondensation it is however imperative to remove monofunctionalized impurities as these will inevitably result in chain-termination and low molecular weights. A suitable purification method is recycling gel permeation chromatography (rGPC).

The oligophenylene monomer 13a can be synthesized in essentially the same way using phencyclone 39 instead of tetraphenylcyclo-pentadienone 11 in the Diels-Alder reaction, according to Scheme 4a.

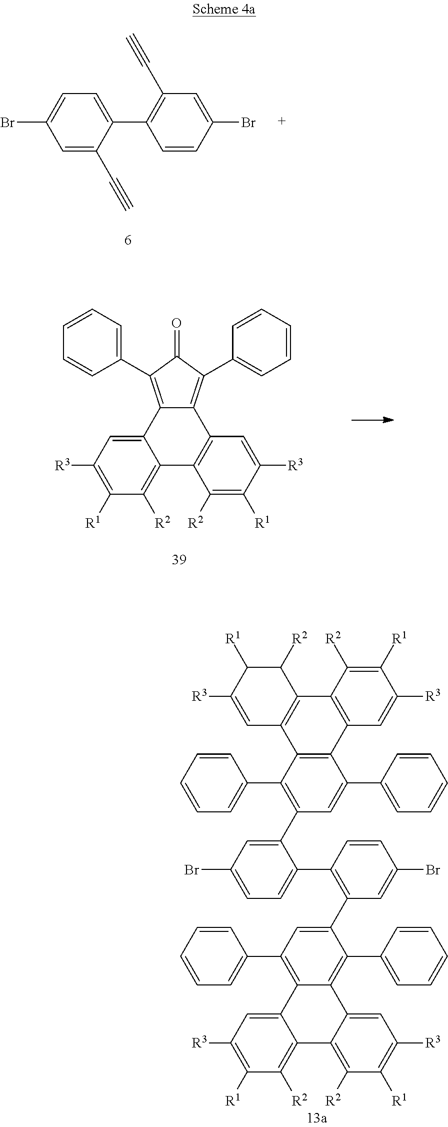

In one aspect of the present invention, oligophenylene monomers of the formula I or II are prepared by Diels-Alder reaction of 4,4′-dibromo-2,2′-diethynyl-1,1′-biphenyl 6 with tetraphenylcyclopentadienone 11 or phencyclone 39, respectively.

As a consequence of Carothers' law, high number-average molecular weights Mₙ are only achieved via polycondensation at high conversion and if at the same time the stoichiometry of the functional groups is strictly maintained.

The purity of all reactants needs to be maximized. Equally, the weighing of both monomer components has to be as precise as possible.

In one further aspect of the present invention, precursors having repeating units V or VI are prepared from oligophenylene monomers of formula I or II, respectively, by copolymerization with 1,4-phenyldiboronic acid or 1,4-phenyldiboronic acid ester. The reaction is generally carried out in solution.

The polymerization of monomers 13 and 13a with e.g. the bis(pinacol) ester of 1,4-phenyldiboronic acid 14 can be carried out by applying standard Suzuki-Miyaura conditions according to Scheme 5, 5a. Both components are placed in a Schlenk tube, which is filled with toluene and a few drops of phase transfer agent Aliquat 336.

High concentrations are favorable for the formation of high molecular weight species during polycondensation. This is due to an enhanced probability of intermolecular coupling events. Aqueous potassium carbonate solution is added as a base. In order to prevent early deactivation of the catalyst, oxygen is removed. Then, tetrakis(triphenylphosphine)palladium(0) is added to the mixture.

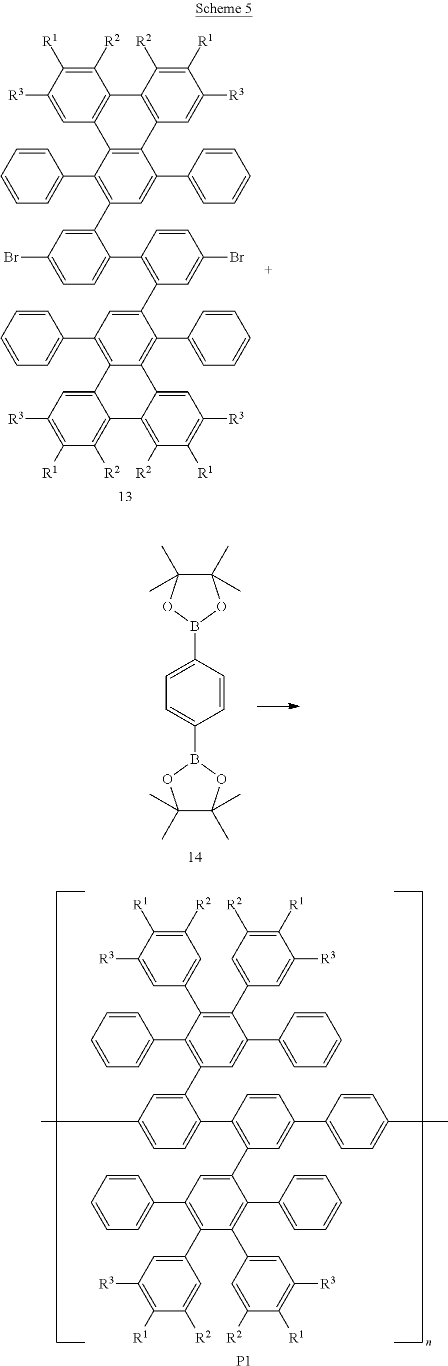

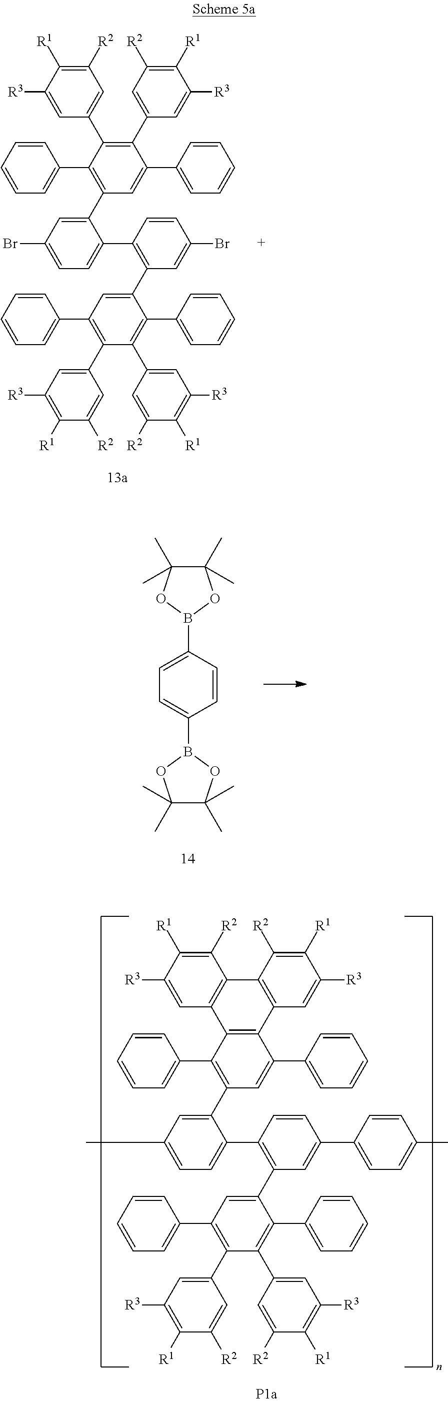

The polymerization is then allowed to proceed for three days at reflux temperature. Afterwards, excess bromobenzene followed by excess phenylboronic acid are added as capping agents.

The preparation of GNRs from the two high-molecular weight precursor P1 and P1a can be performed using ferric chloride as oxidant in a mixture of DCM and nitromethane, both yielding the same GNR1 schematically depicted in FIG. 1. Alternatively, the preparation of GNRs can be carried out using phenyliodine(III) bis(trifluoroacetate) (PIFA) and BF₃ etherate in anhydrous DCM.

In one further aspect of the present invention, GNRs are prepared by cyclodehydrogenation of polymeric precursor P1 and P1a in solution.

The Suzuki-Miyaura protocol can be successfully applied to the synthesis of the laterally extended poly(para-phenylenes) and graphene nanoribbons derived thereof.

However, Suzuki polycondensation reveals several drawbacks:
Due to the sensitivity of A₂B₂-type polycondensation reactions to stoichiometry, the equimolar presence of the two functional groups needs to be precisely controlled. In particular, accurate weighing of small amounts on the milligram scale proved to be challenging.
Aberration from stoichiometry will result in lower molecular weights and shorter lengths of both the poly(para-phenylene) and the derived GNR.
Furthermore, only extended reaction times lead to high molecular weights as a consequence of the underlying kinetics of the polycondensation mechanism.
The bromine atoms of the biphenyl monomer are considerably shielded which might hamper the formation of higher molecular weights due to steric reasons. A more exposed position on the monomer backbone should facilitate polymerization.

Many transition-metal mediated aryl-aryl couplings rely on the addition of an A-functionalized unit to a B-substituted counterpart. In comparison, only a few catalytic protocols are available for efficient AA-type couplings. One of the most versatile methods for the build-up of polymers with a stiff aromatic backbone is the nickel(0) mediated Yamamoto dehalogenation polycondensation. Therefore, the Yamamoto protocol appears a promising tool for the synthesis of high-molecular weight polymeric precursors for GNRs as well. The following points summarize the possible advantages:
For an AA-type polymerization system, only one bifunctionalized component is needed. For this reason, the precise weighing of two components is circumvented. This will result in higher molecular weights and an increase of the GNR length.
The addition of new monomer to the growing polymer chain occurs in a step-wise manner, only AA-type monomer and AA-functionalized chain-ends are present in the reaction mixture.
It is known, that the dehalogenation mechanism mostly leads to non-functionalized chain ends if the reaction is quenched.
Inorganic nickel residues are easily decomposed by acid treatment of the polymer after reaction. The purity of the graphene material if applied as active component in electronic devices is crucial.

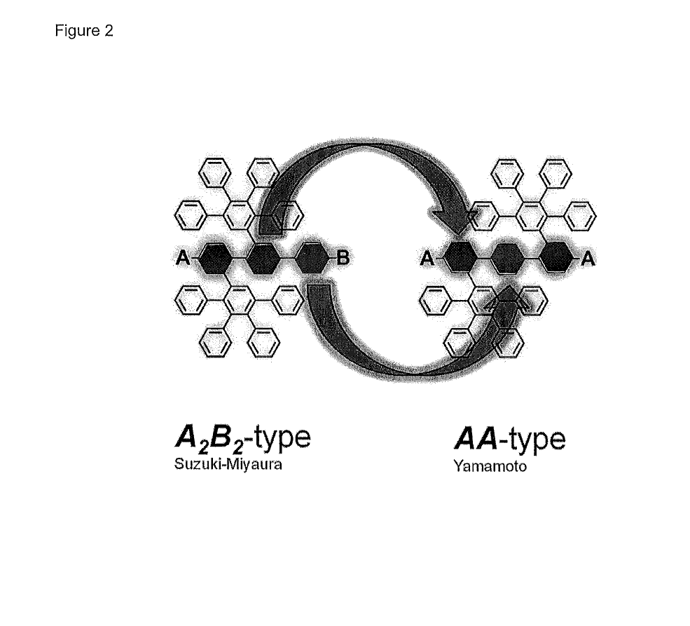

For the Yamamoto polymerization, however, fully symmetric monomers are needed; else a statistical head-tail mixture will result. As depicted in FIG. 2, the repeat unit of the Suzuki-Miyaura system had to be transformed into a new monomer for the Yamamoto approach. This can be done, by “inserting” the benzene ring (red) originating from the BB-type monomer into the biphenyl unit (blue) of the new AA-type monomer. By this, the monomer backbone is extended to a para-terphenyl with the 2,3,4,5-tetraphenylbenzene dendrons attached to its two peripheral benzene rings. Another benefit from this modification is the fact that the two halogen functions are now better accessible as the steric shielding by neighboring benzene rings is reduced in the case of the para-terphenyl geometry.

The connection pattern of repeat unit constitutes an important aspect in the synthesis of GNRs. The periphery will have a strong influence on the final character of the material and can be used to efficiently tune the electronic properties. For steric reasons, the Suzuki-Miyaura system only allows for para-connection of the two monomers. In the case of the Yamamoto approach, also a meta-functionalized oligophenylene monomer is possible thus leading to a kinked backbone chain.

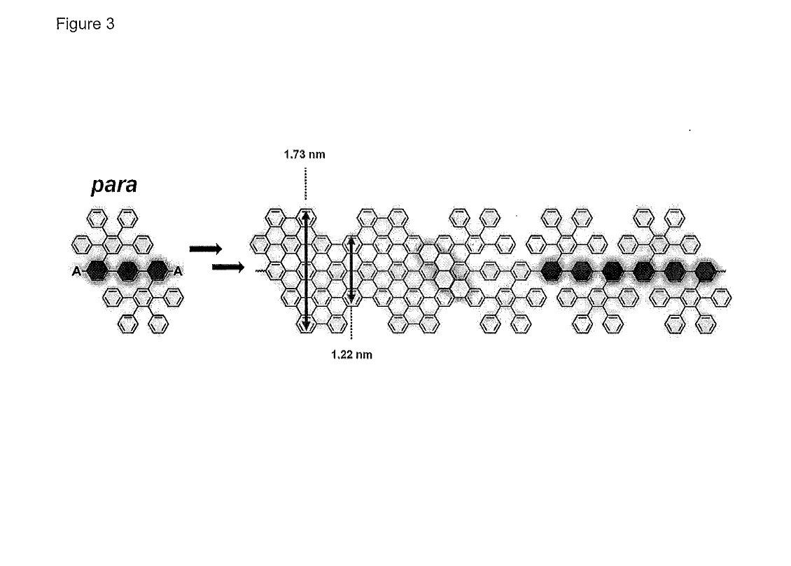

As schematically depicted in FIG. 3, the fusing of two repeat units is achieved by four benzene rings in the case of para-connected GNR2. The width of the nanoribbon varies between 1.73 nm and 1.22 nm (MMFF94s).

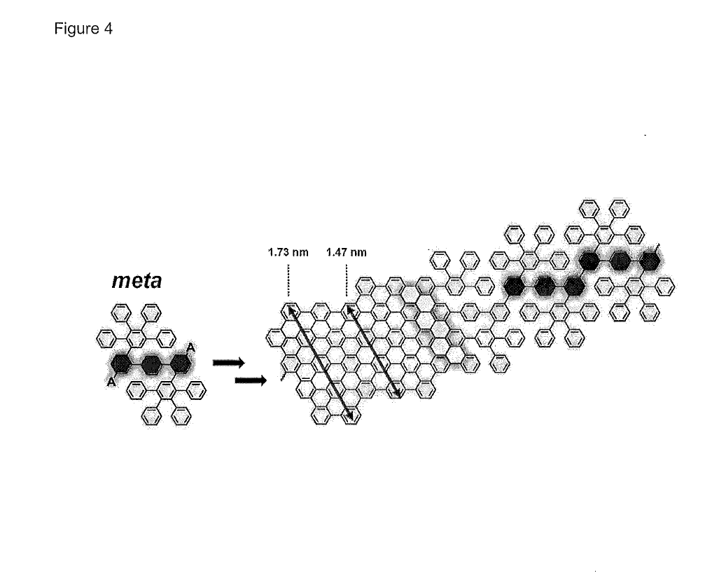

These structural parameters greatly change when a meta-functionalization as in the case of GNR3 is chosen, as shown in FIG. 4. The different connectivity of the building units leads to an enhanced overlap via six aromatic rings. The π-surface of the resulting GNRs is greatly increased further illustrating the power of controlling the structural parameters of graphene materials by precise chemical tailoring.

Due to the induced kink, the armchair-periphery of the molecule is significantly smoothened comparing GNR3 to GNR2, resulting in a maximum lateral extension of 1.73 nm and a minimum value of only 1.47 nm (MMFF94s).

In preferred embodiments, oligophenylene monomers of general formulae IIIa or IIIb are used for the preparation of the polymeric precursor by Yamamoto coupling reaction.

The synthesis of oligophenylene monomers of general formulae IIIa and IIIb can be carried out as summarized below in Schemes 6 to 8.

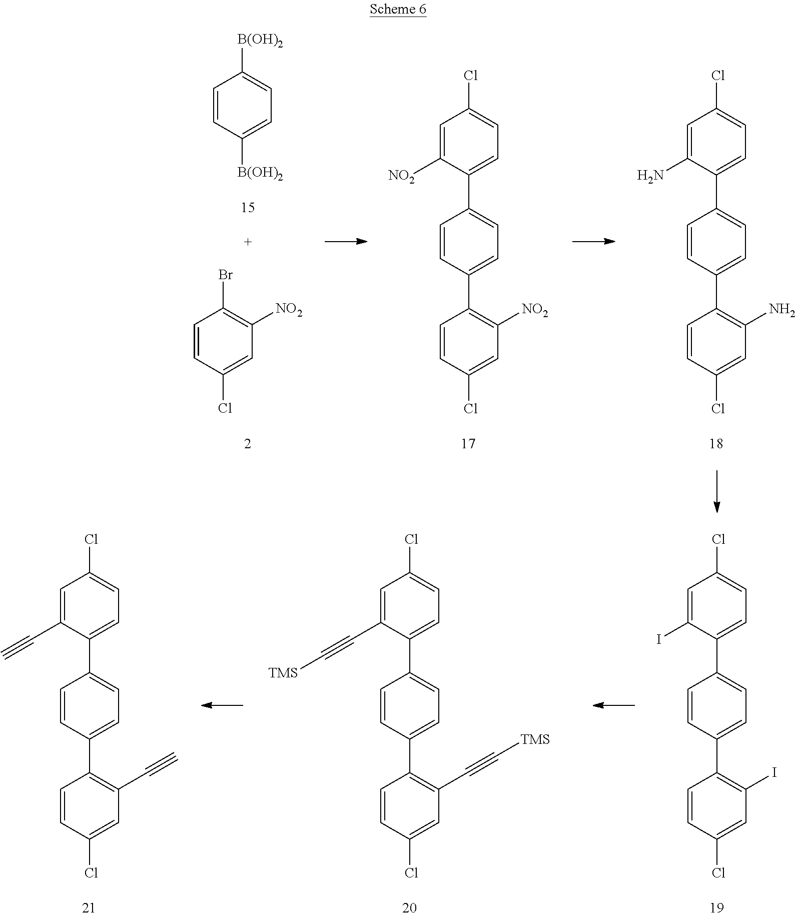

The synthesis of the para-functionalized bisacetylene 21 starts from commercially available 1,4-phenyldiboronic acid 15 and 1-bromo-4-chloro-2-nitrobenzene 16. Suzuki-Miyaura coupling of both components yields the functionalized para-terphenyl 17. The desired compound precipitates during the course of the reaction. Subsequently, the two nitro-groups are converted into the corresponding amine functions by reduction with hydrogen gas in the presence of carbon-supported palladium(0).

The diamine 18 is converted into 4,4″-dichloro-2,2″-diiodo-1,1′:4′,1″-terphenyl 19 by double Sandmeyer reaction. Two-fold Sonogashira-Hagihara cross-coupling with trimethylsilyl acetylene in the presence of bis(triphenylphosphine)palladiumchloride(II) and copper iodide gives the protected bisacetylene 20. The deprotection of this compound can be achieved by the aforementioned method using potassium carbonate as base. Remaining impurities of mono-substituted by-product can be removed by final column chromatography of 21.

The meta-functionalized bisacetylene 26 can be prepared in a similar fashion using a closely related synthetic sequence. However the initial Suzuki-Miyaura reaction works also well in the presence of free amine groups. By coupling 2-bromo-4-chloroaniline 22, 5,5″-dichloro-[1,1′:4′,1″-terphenyl]-2,2″-diamine 23 is prepared. The compound is directly converted into 24. This compound is then transformed into compound 26 using identical synthetic conditions as described above (Scheme 7).

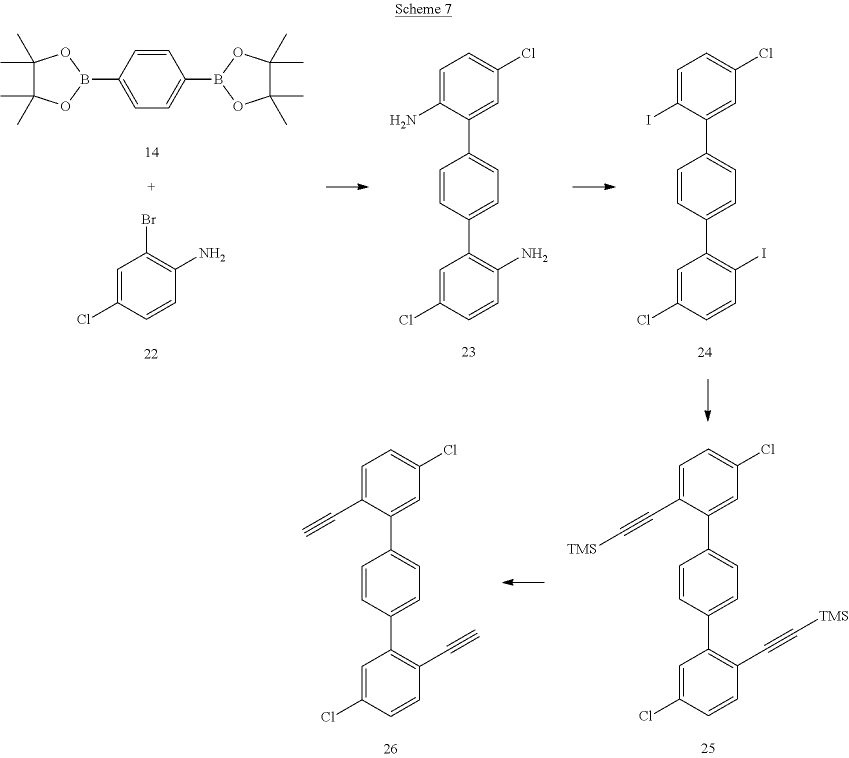

Both functionalized para-terphenyls show a strong tendency to crystallize which can be attributed to the rigid nature of the molecules and the two peripheral ethinyl groups for which a high packing tendency is known.

In the final step, Diels-Alder reaction of 21 and 26 with alkyl-functionalized tetraphenylcyclopentadienone 37 is used for the preparation of the corresponding oligophenylene monomers 27 and 28, respectively (Scheme 8). The reactions can be carried out under microwave irradiation in ortho-xylene at 160° C.

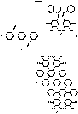

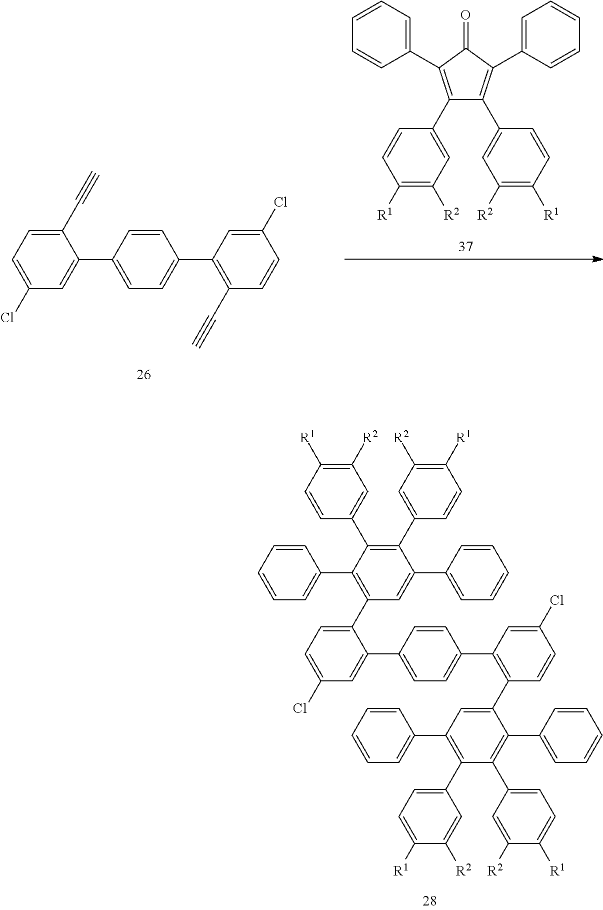

The two dendronized terphenyl monomers 27 and 28 can be isolated by rGPC as colorless oils that solidify upon standing.

The new para-terphenyl geometry of monomers 27 and 28 has not been reported in the preparation of nanographene materials so far.

In one further aspect of the present invention, oligophenylene monomers of general formulae IIIa and IIIb, wherein X, Y═Cl, are prepared by Diels-Alder reaction of the dichloro-bisacetylenes 21 and 26, respectively, with tetraphenylcyclopentadienone 37. More generally, oligophenylene monomers of general formulae IIIa and IIIb, wherein X, Y=halogene, are prepared from tetraphenylcyclopentadienone and the respective dihalo-bisacetylenes.

In a further aspect of the present invention, graphene nanoribbons are prepared by cyclodehydrogenation of polymeric precursors in a solution process. The polymeric precursors are obtained from the polyphenylene monomers as described above.

With the monomers 27 and 28 available their polycondensation can be carried out using the standard Yamamoto protocol (according to Scheme 9). The reaction can be carried out e.g. in an overall 3/1 mixture of toluene/DMF. The catalyst can be prepared from a stoichiometric mixture of bis(cyclooctadiene)nickel(0), 1,5-cyclooctadiene and 2,2′-bipyridine e.g. in toluene/DMF. The reaction can likewise be carried out using the dibromo- instead of the dichloro-compound.

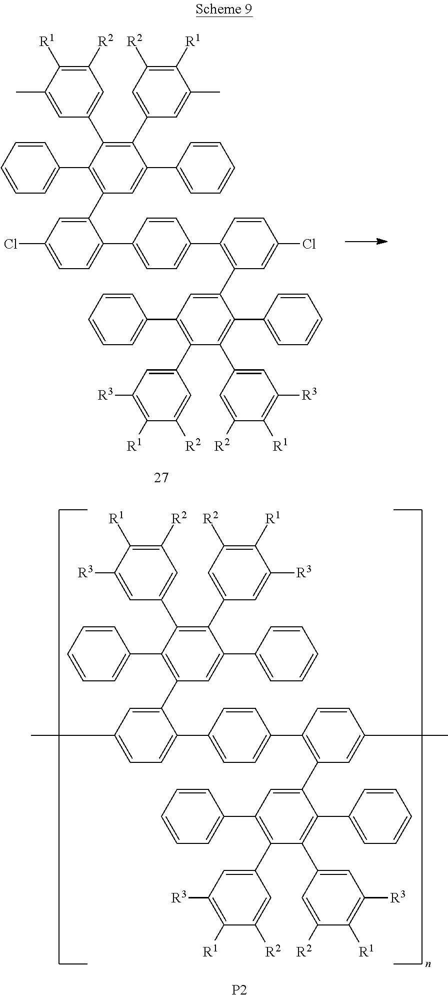

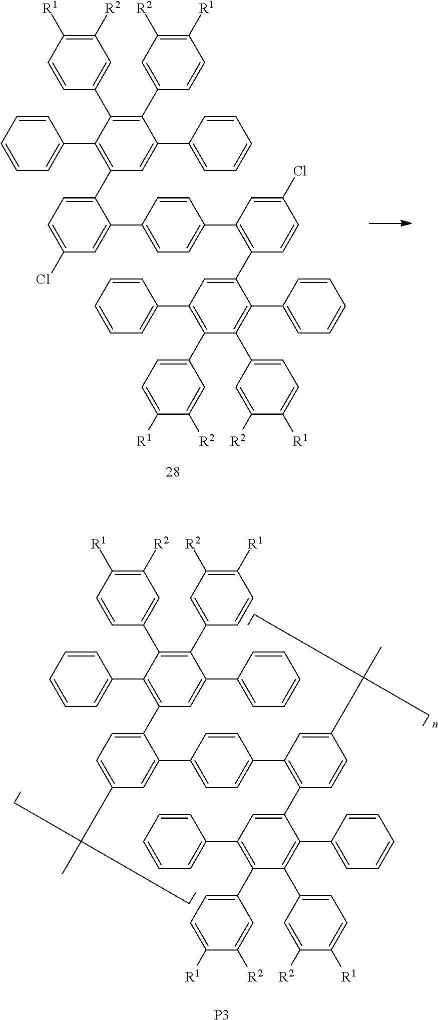

The quenching of the reaction and the decomposition of nickel residues can be achieved by carefully dropping the reaction mixture into dilute methanolic hydrochloric acid. A white precipitate instantly formed which can be collected by filtration. The material can be re-dissolved in DCM, filtered and re-precipitated. The number of repeating units n varies in general from 5 to 100 preferably from 20 to 50.

In a particular aspect of the present invention, GNRs are prepared from precursors P2 or P3 by cyclodehydrogenation in solution in the presence of an oxidant (Scholl reaction).

The preparation of GNRs from the two high-molecular weight precursors P2 and P3 can be performed using ferric chloride as oxidant in a mixture of DCM and nitromethane. Alternatively, the preparation of GNRs can be carried out using phenyliodine(III) bis(trifluoroacetate) (PIFA) and BF₃ etherate in anhydrous DCM. Graphitic insoluble materials are obtained in quantitative yield. The corresponding materials will be referred to as GNR2 and GNR3 in the following.

In general, the molecular weight of the GNRs obtained varies from 10 000 to 200 000, preferably from 30 000 to 80 000.

Covalently bonded two-dimensional molecular arrays can be efficiently studied by STM techniques. Examples of surface-confined covalent bond formation involve Ullmann coupling, imidization, crosslinking of porphyrins and oligomerization of heterocyclic carbenes and polyamines. A chemistry-driven protocol for the direct growth of GNRs and graphene networks on surfaces has been very recently established by the groups of Mallen (MPI-P Mainz, Germany) and Fasel (EMPA Dübendorf, Switzerland). Without being bound by theory it can be concluded from these studies that the nanoribbon formation on the metal surface proceeds via a radical pathway. After deposition of the functionalized monomer on the surface via UHV sublimation instant dehalogenation is believed to occur. This generates biradical species that diffuse on the surface and couple to each other resulting in the formation of carbon-carbon bonds. These radical addition reactions proceed at intermediate thermal levels (200° C.) and are the prerequisite for the subsequent cyclodehydrogenation at higher temperatures (400° C.). Only if polymeric species of sufficient molecular weight are formed during the first stage, the full graphitization of the molecules will proceed subsequently with the thermal desorption of the material from the surface being avoided.

For UHV STM-assisted surface polymerization and cyclodehydrogenation, functional monomers of high rigidity and planarity are needed which assist in the flat orientation on the metal substrate. Also, the method allows for the topological tailoring of the GNRs as their shape it is determined by the functionality pattern and geometry of the precursor monomers.

In a further aspect of the present invention, graphene nanoribbons are prepared by direct growth of the graphene nanoribbons on surfaces by polymerization of the monomers as described above and cyclodehydrogenation.

In one particular preferred embodiment, oligophenylene monomers of general formula IVa or IVb are used for the preparation of the polymeric precursor by Yamamoto coupling reaction. In some particular preferred embodiments, monomers IVa or IVb are used in the direct growth of GNRs on surfaces by polymerization of the monomers and cyclodehydrogenation.

As an alternative to monomers 27 and 28 used for the solution-based fabrication of GNR2 and GNR3, the two analogous oligophenylene monomers 29 and 30 can be used. The use of the rigid building block phencyclone 39 in the Diels-Alder reaction with the bisacetylenes 21 and 26 results in the formation of pre-planarized dendrons that contain a triphenylene moiety. The decrease of conformational flexibility is one of the requirements for the surface-assisted approach. The two oligophenylenes 29 and 30 can be obtained by the established Diels-Alder route according to Scheme 10. After standard column chromatography both monomers can be purified by means of rGPC. The purity can be confirmed by MALDI-TOF and NMR spectroscopy.

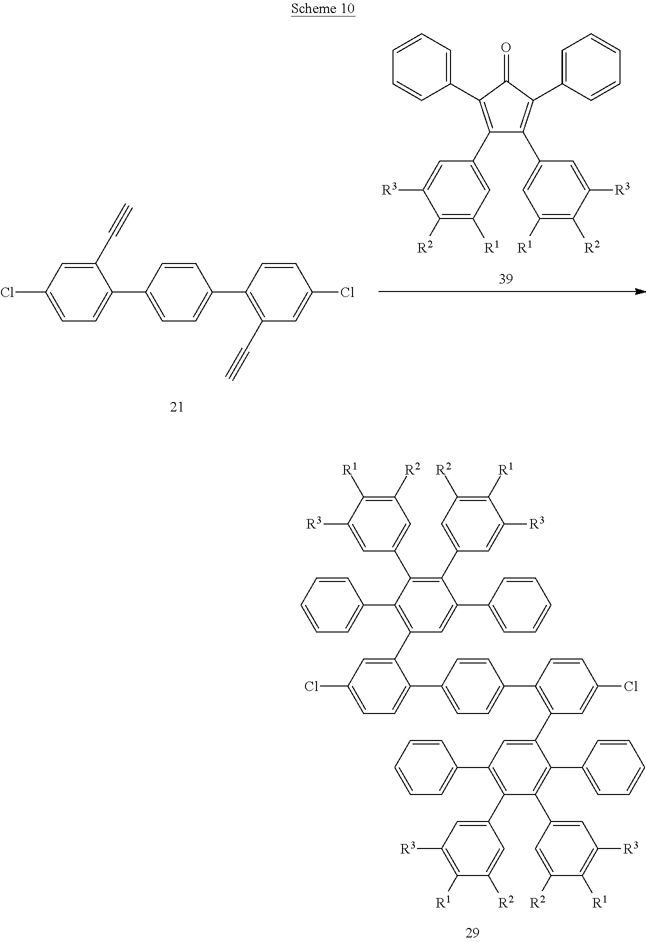

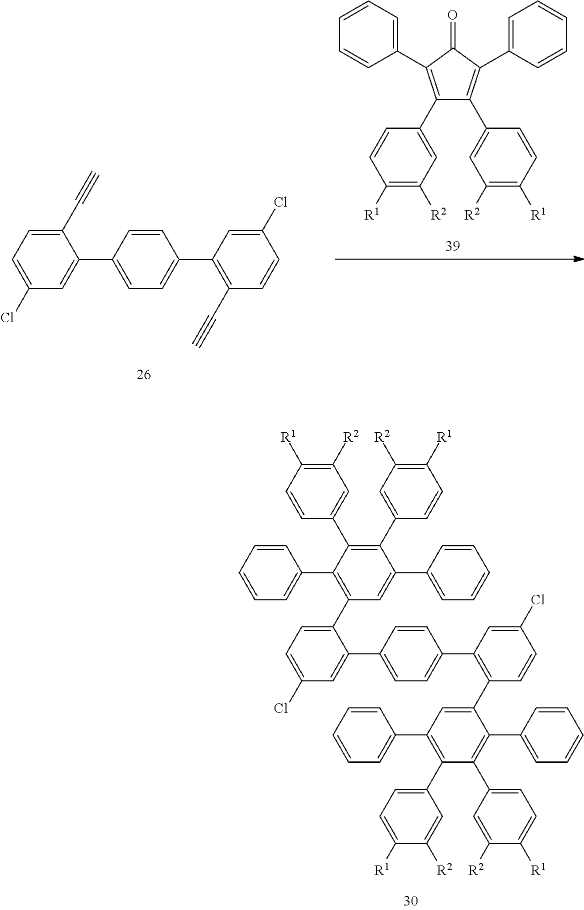

In one further aspect of the present invention, oligophenylene monomers of general formulae IVa or IVb, wherein X, Y═Cl, are prepared by Diels-Alder reaction of the dichloro-bisacetylenes 21 and 26, respectively, with phencyclone 39. More generally, the oligophenylene monomers of general formulae IVa or IVb, wherein X, Y=halogene, are prepared from phencyclone and the respective dihalo-bisacetylenes.

Despite their molecular weights of 1056 g/mol, both molecules can be successfully deposited on various metal substrates at a temperature of 330° C.

In one particular preferred embodiment, oligophenylene monomers of general formula IVa, wherein X═Br, is used in the direct growth of GNRs on surfaces by polymerization of the monomers and cyclodehydrogenation.

Increasing the halogen reactivity may lead to a more efficient polymerization and thereby result in an increase of the molecular weight. One of the key steps of the surface protocol is the formation of a radical at the moment where the monomer contacts the metal substrate from the gas phase. It can be assumed that decreasing the strength of the carbon-halogen bond will efficiently support the formation of the active site and thus lead to a more efficient polymerization. Additionally, high molecular weight species will progressively lose their surface mobility which could also be beneficial for the successive planarization of the polymeric structure. Based on these considerations the two chlorine atoms of 29 are preferably exchanged by two bromine atoms. The synthesis of the analogous dibromooligophenylene 36 is summarized in Schemes 11 and 12.

Starting from 4,4″-dibromo-2,2″-dinitro-1,1′:4′,1″-terphenyl 31, he synthesis of the functionalized bisacetylene 35 can be achieved using the established synthetic route according to Scheme 11.

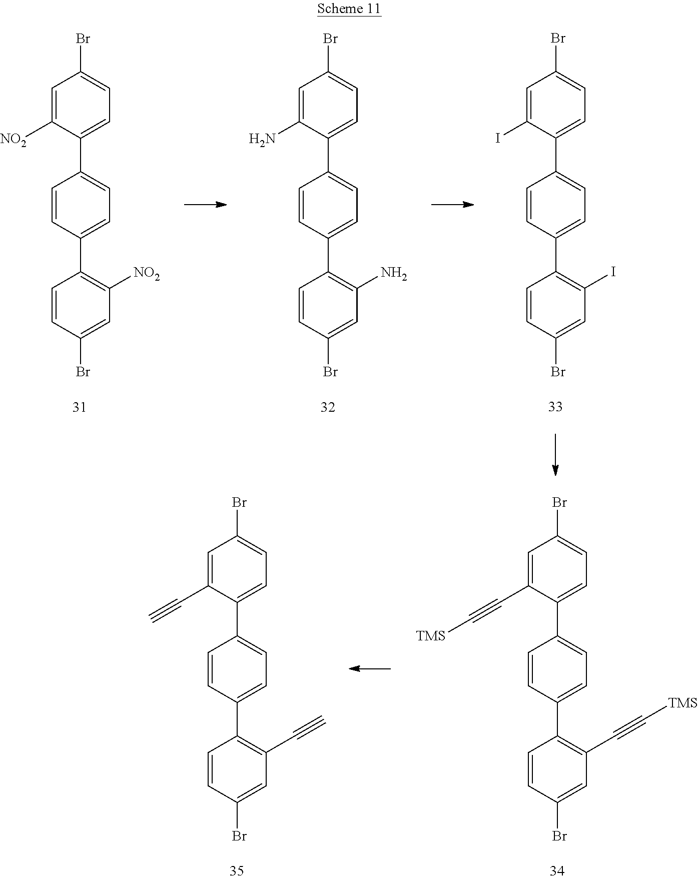 [SMILES: O=[N+]([O-])c1cc(Br)ccc1-c1ccc(-c2ccc(Br)cc2[N+](=O)[O-])cc1 ; Nc1cc(Br)ccc1-c1ccc(-c2ccc(Br)cc2N)cc1 ; Brc1ccc(-c2ccc(-c3ccc(Br)cc3I)cc2)c(I)c1 ; C#Cc1cc(Br)ccc1-c1ccc(-c2ccc(Br)cc2C#C)cc1 ; C[Si](C)(C)C#Cc1cc(Br)ccc1-c1ccc(-c2ccc(Br)cc2C#C[Si](C)(C)C)cc1]

The difference in reactivity of the iodine and bromine atoms of 33 at room temperature made the synthesis of the protected bisacetylene 34 possible by the regioselective Sonogashira-Hagihara cross-coupling with trimethylsilyl acetylene.

The bisacetylene 35 is then again reacted with phencyclone 39 to give the rigidified oligophenylene precursor 36 having enhanced reactivity towards surface polymerization according to Scheme 12.

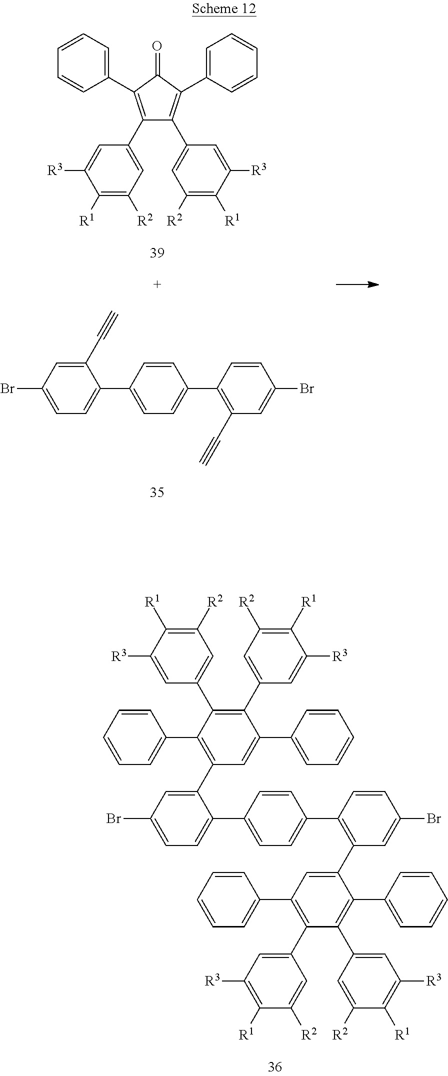

In one further aspect of the present invention, oligophenylene monomers of general formula IVa, wherein X═Br, are prepared by Diels-Alder reaction of bisacetylenes 35 with phencyclone 39.

GNRs can be prepared from monomers 29, 30 and 31 by UHV STM-assisted surface polymerization and cyclodehydrogenation.

In one further aspect of the present invention, GNRs are prepared form monomers IVa or IVb by direct growth of the GNRs on surfaces by polymerization of the monomers and cyclodehydrogenation.

In alternative embodiments, oligophenylene monomers of general formulae A-F can also be obtained via Suzuki or Stille coupling reactions, as exemplified below by Schemes 13-19.

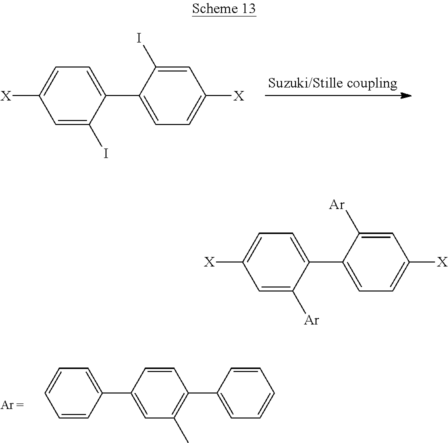

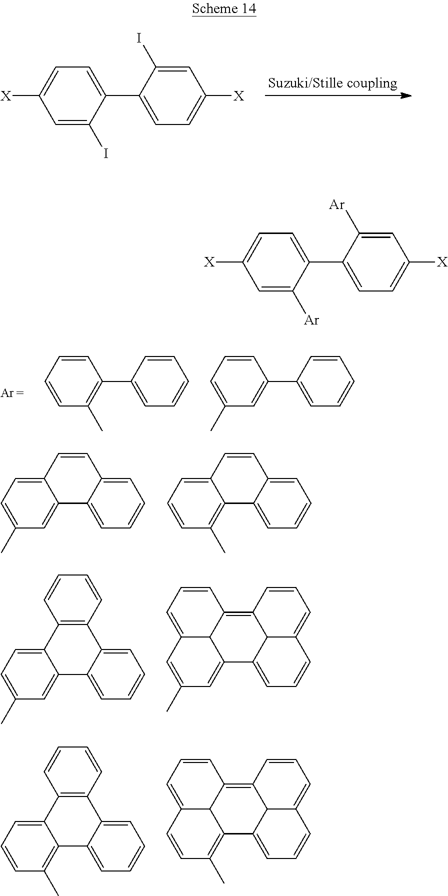

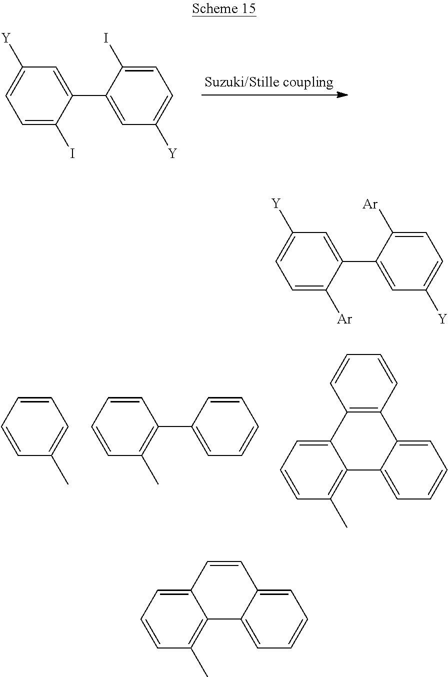

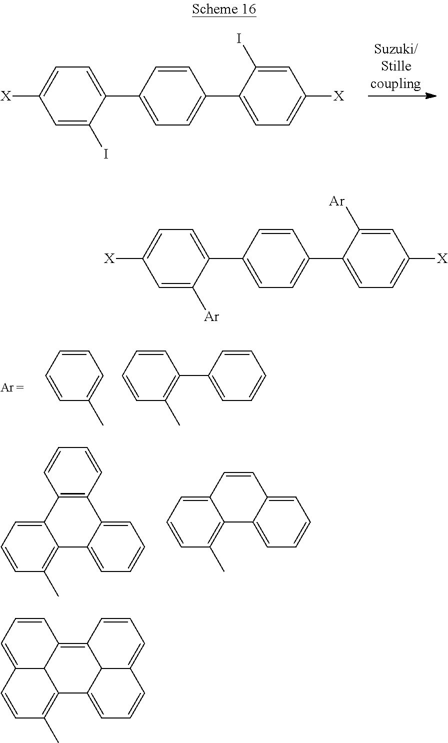

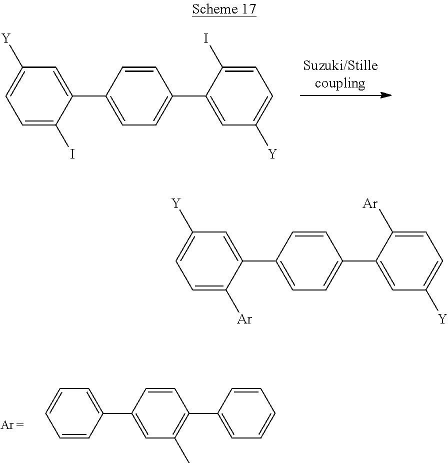

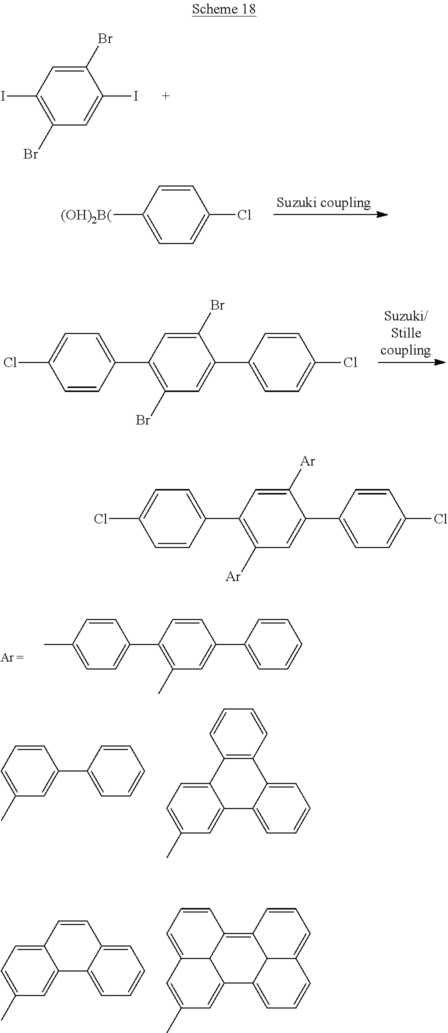

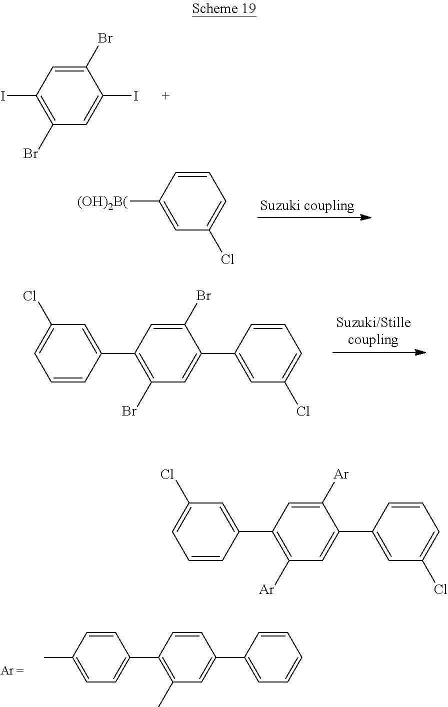

The invention is illustrated in more detail by the following examples.

### EXAMPLES

FIGS. 1-8 show:

Structural design of A₂B₂ system GNR1 (FIG. 1)

Schematic representation illustrating the monomer design of a suitable AA-type system from the A₂B₂ system (FIG. 2)

Schematic representation of Yamamoto-based graphene nanoribbons GNR2 (FIG. 3)

Schematic representation of Yamamoto-based graphene nanoribbons GNR3 (FIG. 4)

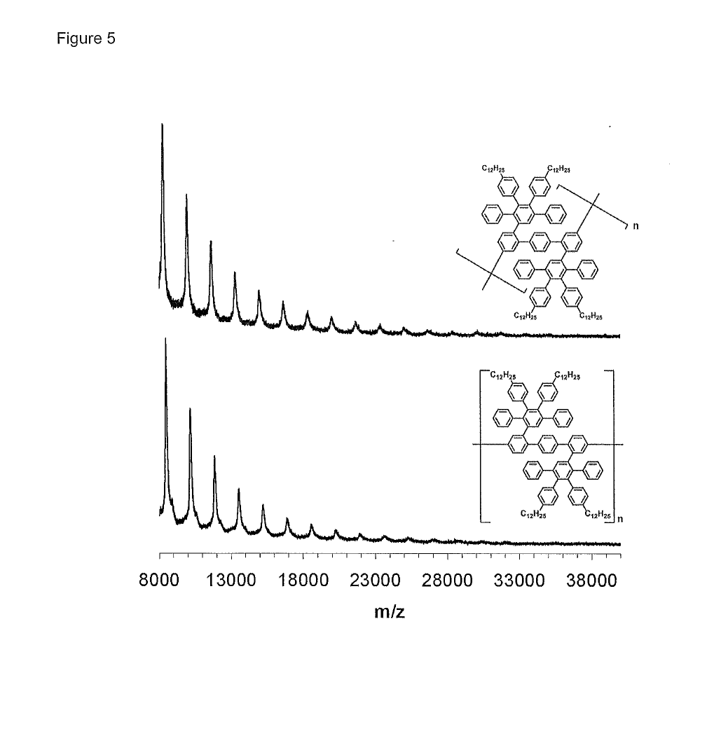

MALDI-TOF spectra of P1 and P2 (FIG. 5)

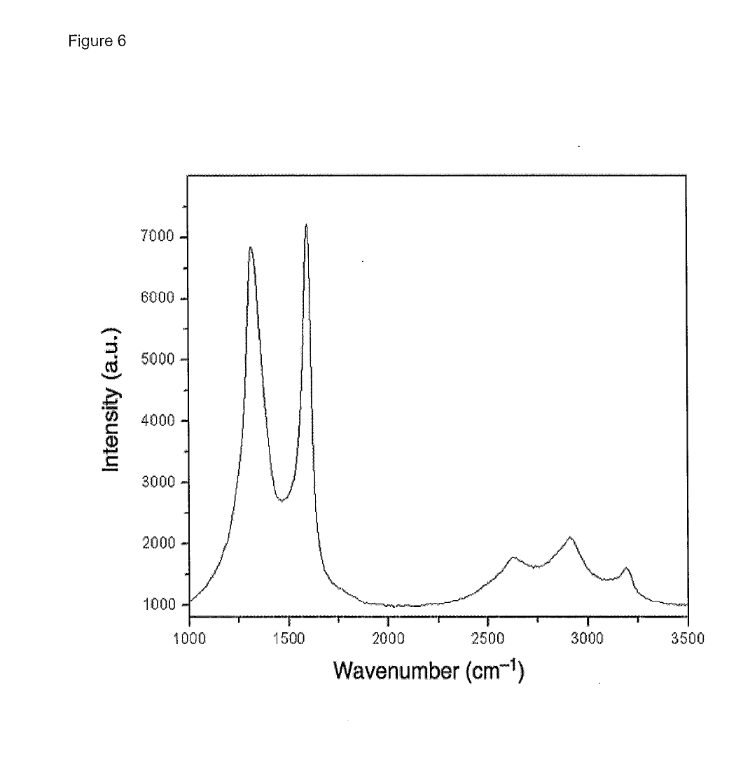

Raman Spectrum of GNR2 (FIG. 6)

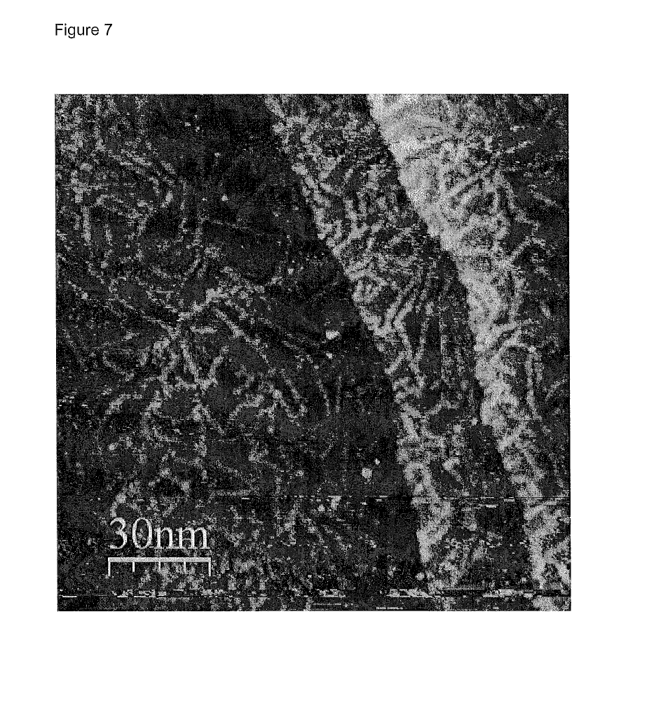

STM image of 36 after deposition and annealing on Au (111) (FIG. 7)

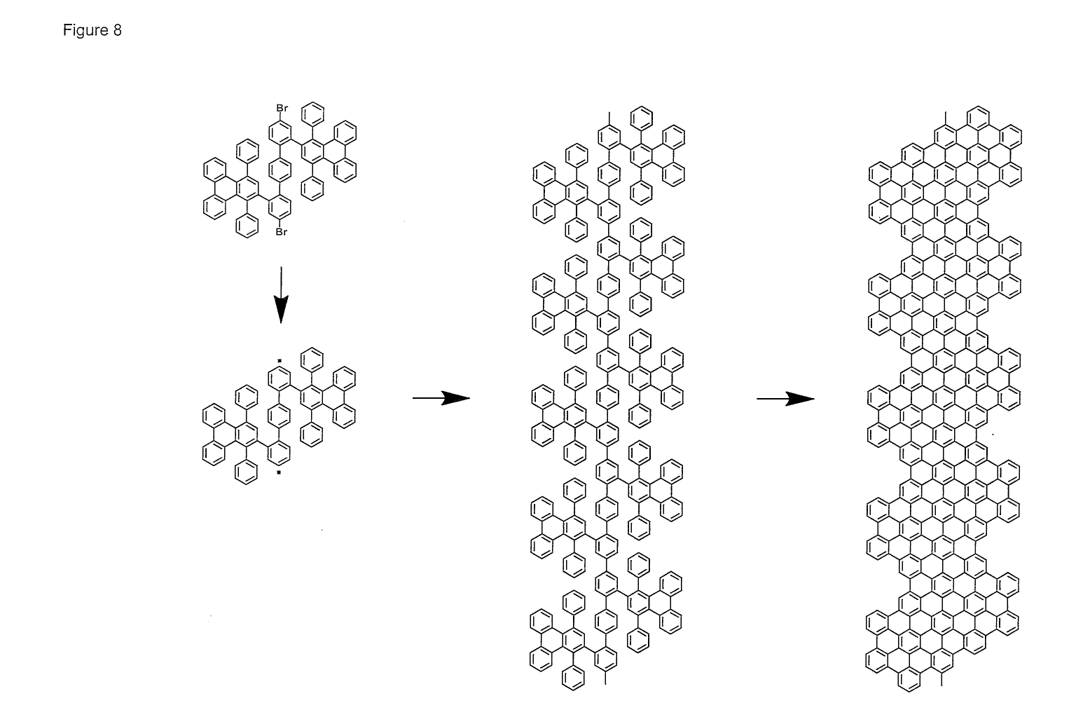

Polymerization and cyclodehydrogenation pathway for the surface preparation of GNR (FIG. 8)

### Example 1A Preparation of 4,4″-Dichloro-2,2″-dinitro-1,1′:4′,1″-terphenyl (3)

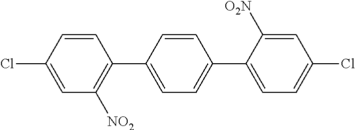 [SMILES: O=[N+]([O-])c1cc(Cl)ccc1-c1ccc(-c2ccc(Cl)cc2[N+](=O)[O-])cc1]

15.00 g (63.44 mmol) 1-bromo-4-chloro-2-nitrobenzene and 5.00 g (30.17 mmol) 1,4-phenyldiboronic acid were dissolved in 215.0 ml of dioxane. Then, a few drops of Aliquat 336 and 85.0 ml of an aqueous K₂CO₃ (2 M) were added. After degassing by argon bubbling, 0.70 g (0.61 mmol) of tetrakis(triphenylphosphine)palladium(0) were added. The reaction mixture was heated to reflux for 24 h. After cooling, the reaction mixture was poured on ice. 10.35 g (26.55 mmol) of a yellow precipitate which formed were collected, washed with methanol and used without further purification for the next step (88%).

¹H NMR (250 MHz, CD₂Cl₂): δ 7.92 (d, J=2.1, 2H), 7.67 (dd, J=2.2, 8.3, 2H), 7.48 (d, J=8.3, 2H), 7.38 (s, 4H).

¹³C NMR (75 MHz, CD₂Cl₂): δ 149.89, 137.36, 134.88, 134.60, 133.79, 133.27, 128.89, 124.98.

MS (FD, 8kV): m/z (%)=387.1 (100.0%, M⁺), (calc. C₁₈H₁₀Cl₂N₂O₄=389.91 g/mol).

Elemental Analysis: found 56.56% C, 3.09% H, 6.53% N—calc. 55.55% C, 2.59% H, 7.20% N.

### Example 1B Preparation of 4,4″-Dichloro-[1,1′:4′,1″-terphenyl]-2,2″-diamine 18

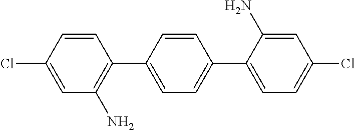 [SMILES: Nc1cc(Cl)ccc1-c1ccc(-c2ccc(Cl)cc2N)cc1]

5.00 g (12.85 mmol) 17 and 0.70 g of palladium on carbon (10 wt %) were suspended in 200.0 ml of THF. The reaction mixture was evacuated after what a balloon filled with hydrogen gas was connected. The reaction mixture was heated to 50° C. for 24 h under vigorous stirring and monitored by thin-layer chromatography. With the consumption of the starting compound the reaction mixture turned homogenous. The crude product was purified by column chromatography (hexane/ethyl acetate=7/3) to yield 3.89 g (11.82 mmol) of 18 as a yellow solid in 92%.

¹H NMR (300 MHz, CD₂Cl₂): δ 7.40 (s, 4H), 6.96 (d, J=6.4, 2H), 6.69 (dd, J=2.0, 6.5, 4H), 3.88 (s, 4H).

¹³C NMR (75 MHz, CD₂Cl₂): δ 145.66, 138.21, 134.42, 132.00, 130.04, 125.98, 118.82, 115.57.

MS (FD, 8kV): m/z (%)=327.3 (100.0%, M⁺), (calc. C₁₈H₁₀Cl₂N₂O₄=329.22 g/mol).

Elemental Analysis: found 63.87% C, 4.39% H, 7.15% N—calc. 65.67% C, 4.29% H, 8.51% N.

### Example 1C Preparation of 4,4″-Dichloro-2,2″-diiodo-1,1′:4′,1″-terphenyl 19

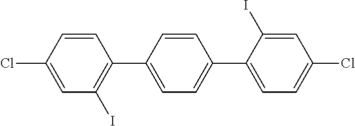 [SMILES: Clc1ccc(-c2ccc(-c3ccc(Cl)cc3I)cc2)c(I)c1]

3.00 g (9.11 mmol) of 18 were suspended in 20.0 ml of water. Then, 12.0 ml of concentrated hydrochloric acid were added under cooling. At a temperature of −5° C., 10.0 ml of an aqueous solution containing 1.56 g (22.58 mmol) sodium nitrite were added dropwise. During this procedure, the color of the reaction mixture changed from yellow to dark brown. Subsequently, 30.0 ml of an aqueous solution containing 15.29 g (91.18 mmol) potassium iodide were added dropwise while maintaining the temperature below 0° C. After the addition, the reaction was allowed to proceed for 1 h at room temperature. After extraction with DCM, treatment with an aqueous solution of sodium thiosulfate and removal of the solvent under reduced pressure the crude product was purified by column chromatography (hexane/ethyl acetate=20/1) to yield 1.96 g (3.55 mmol) of 19 in 39% as a yellowish solid

¹H NMR (300 MHz, CD₂Cl₂): δ 8.00 (d, J=2.1, 2H), 7.43 (dd, J=2.0, 8.5, 2H), 7.40 (s, 4H), 7.31 (d, J=8.2, 2H).

¹³C NMR (75 MHz, CD₂Cl₂): δ 145.27, 143.16, 139.39, 134.20, 131.21, 129.53, 128.99, 98.77.

MS (FD, 8kV): m/z (%)=549.1 (100.0%, M⁺), (calc. C₁₈H₁₀Cl₂I₂=550.99 g/mol).

Elemental Analysis: found 40.55% C, 2.13% H—calc. 39.24% C, 1.83% H.

### Example 1D Preparation of 4,4″-Dichloro-2,2″-diethynyl-1,1′:4′,1″-terphenyl 21

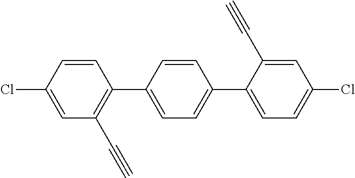 [SMILES: C#Cc1cc(Cl)ccc1-c1ccc(-c2ccc(Cl)cc2C#C)cc1]

0.50 g (0.91 mmol) of 19 were mixed with 20.0 mg (0.11 mmol) of copper(II) iodide and 15.0 ml of triethylamine. After degassing by argon bubbling, 40.0 mg (0.06 mmol) of bis(triphenylphosphine)palladium(II) dichloride and 0.27 ml (1.36 mmol) of (trimethylsilyl)acetylene were added. The reaction mixture was stirred at room temperature for 24 h under an inert atmosphere and monitored by thin-layer chromatography. The reaction mixture was filtered over a silica pad (DCM) to remove inorganic residues.

The product thus obtained (0.40 g, 0.82 mmol, 90%) was then dissolved in a mixture of 50.0 ml THF and 50.0 ml methanol. Then, 0.70 g (5.07 mmol) potassium carbonate was added and the reaction mixture was stirred at room temperature for 24 h. The crude product was purified by column chromatography (hexane/ethyl acetate=9/1) to yield 0.18 g (0.53 mmol) of 19 in 64%.

¹H NMR (300 MHz, CD₂Cl₂): δ 7.65 (s, 4H), 7.63 (d, J=1.8, 2H), 7.44 (dd, J=2.1, 8.4, 2H), 7.39 (dd, J=0.5, 8.4, 2H), 3.20 (s, 2H).

¹³C NMR (75 MHz, CD₂Cl₂): δ 142.82, 139.19, 134.04, 133.51, 131.50, 129.95, 129.48, 122.51, 82.24, 81.99.

MS (FD, 8kV): m/z (%)=345.5 (100.0%, M⁺), (calc. C₂₂H₁₂Cl₂=347.24 g/mol).

Elemental Analysis: found 75.79% C, 4.26% H—calc. 76.10% C, 3.48% H.

### Example 1E Preparation of 4′″,5′-Dichloro-2,2″″,5,5″″-tetraphenyl-3,3″″,4,4″″-tetra(4-dodecylphenyl)-1,1′:2′,1″:4″,1′″:2′″,1″″-quinquephenyl 27

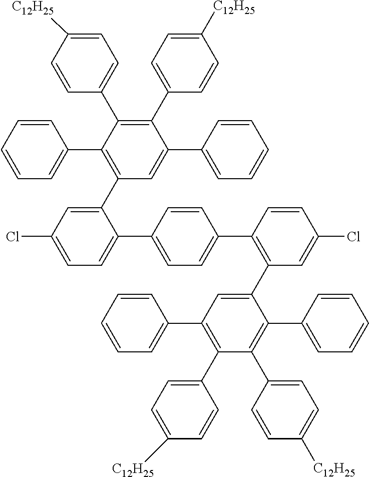

0.14 g (0.40 mmol) 21 and 0.70 g (0.97 mmol) 37 were placed in a microwave vessel. Then, 8.0 ml of ortho-xylene were added and the reaction mixture was degassed by argon bubbling. The reaction vessel was sealed, placed in a microwave reactor and heated to 160° C. at 300 W for 24 h with activated cooling. The crude product was pre-purified by column chromatography (hexane/ethyl acetate=9/1). Further purification was achieved by preparative gel permeation chromatography (chloroform) to yield 0.59 g (0.34 mmol) of 27 in 85% as a transparent oil which solidified upon standing.

¹H NMR (700 MHz, THF): δ 7.50-7.40 (m, 4H), 7.25 (t, J=12.2, 2H), 7.13 (t, J=7.5, 2H), 7.07 (m, 10H), 6.92-6.40 (m, 29H), 6.01-5.80 (d, J=73.9, 1H), 2.38 (t, J=7.5, 4H), 2.28 (t, J=7.3, 4H), 1.43 (p, 4H), 1.36 (p, 4H), 1.32-1.06 (m, 72H), 0.89 (t, J=7.1, 12H).

¹³C NMR (75 MHz, THF): δ 143.24, 142.98, 141.66, 141.16, 140.86, 140.74, 140.32, 140.18, 139.91, 139.79, 139.72, 138.69, 138.51, 133.23, 132.49, 132.33, 132.09, 130.94, 129.98, 128.41, 128.24, 127.86, 127.52, 127.37, 127.07, 126.20, 36.36, 36.29, 33.05, 32.38, 32.32, 30.86, 30.80, 30.65, 30.50, 30.03, 29.95, 29.83, 23.62, 14.65.

MS (FD, 8kV): m/z (%)=1731.6 (100.0%, M⁺), (calc. C₁₂₆H₁₄₈Cl₂=1733.43 g/mol).

Elemental Analysis: found 85.16% C, 9.21% H—calc. 87.30% C, 8.61% H (see general remarks “7.2.4 Elemental Combustion Analysis”).

### Example 2A Preparation of 5,5″-Dichloro-[1,1′:4′,1″-terphenyl]-2,2″-diamine 23

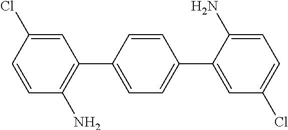 [SMILES: Nc1ccc(Cl)cc1-c1ccc(-c2cc(Cl)ccc2N)cc1]

4.20 g (20.34 mmol) 2-bromo-4-chloroaniline and 3.05 g (9.25 mmol) 1,4-phenyldiboronic acid bis(pinacol) ester were dissolved in 180.0 ml of dioxane. Then, a few drops of Aliquat 336 and 75.0 ml of an aqueous K₂CO₃ (2 M) were added. After degassing by argon bubbling, 0.35 g (0.30 mmol) of tetrakis-(triphenylphosphine)palladium(0) were added. The reaction mixture was heated to reflux for 24 h. The crude product was purified by column chromatography (hexane/ethyl acetate=7/3) to yield 2.41 g (7.31 mmol) of 23 as a yellow solid in 79%.

¹H NMR (300 MHz, CD₂Cl₂): δ 7.52 (s, 4H), 7.12 (dd, J=2.1, 10.1, 4H), 6.72 (dd, J=0.9, 7.9, 2H), 3.88 (s, 4H).

¹³C NMR (75 MHz, CD₂Cl₂): δ 143.21, 138.25, 130.36, 130.01, 128.81, 128.77, 123.30, 117.27.

MS (FD, 8kV): m/z (%)=327.3 (100.0%, M⁺), (calc. C₁₈H₁₀Cl₂N₂O₄=329.22 g/mol).

Elemental Analysis: found 65.65% C, 4.57% H, 7.76% N—calc. 65.67% C, 4.29% H, 8.51% N.

### Example 2B Preparation of 5,5″-Dichloro-2,2″-diiodo-1,1′:4′,1″-terphenyl 24

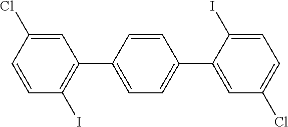 [SMILES: Clc1ccc(I)c(-c2ccc(-c3cc(Cl)ccc3I)cc2)c1]

2.00 g (6.07 mmol) of 23 were suspended in 15.0 ml of water. Then, 8.0 ml of concentrated hydrochloric acid were added under cooling. At a temperature of −5° C., 7.0 ml of an aqueous solution containing 1.04 g (15.05 mmol) sodium nitrite were added dropwise. During this procedure, the color of the reaction mixture changed from yellow to dark brown. Subsequently, 20.0 ml of an aqueous solution containing 10.19 g (60.79 mmol) potassium iodide were added dropwise while maintaining the temperature below 0° C. After the addition, the reaction was allowed to proceed for 1 h at room temperature. After extraction with DCM, treatment with an aqueous solution of sodium thiosulfate and removal of the solvent under reduced pressure the crude product was purified by column chromatography (hexane/ethyl acetate=8/2) to yield 1.40 g (3.55 mmol) of 24 in 42% as a yellowish solid

¹H NMR (300 MHz, CD₂Cl₂): δ 7.91 (d, J=8.5, 2H), 7.41 (s, 4H), 7.39 (d, J=2.5, 2H), 7.08 (dd, J=2.6, 8.5, 2H).

¹³C NMR (75 MHz, CD₂Cl₂): δ 148.20, 143.29, 141.26, 135.03, 130.62, 129.65, 129.49, 96.09.

MS (FD, 8kV): m/z (%)=549.1 (100.0%, M⁺), (calc. C₁₃H₁₀Cl₂I₂=550.99 g/mol).

Elemental Analysis: found 40.60% C, 2.22% H—calc. 39.24% C, 1.83% H.

### Example 2C Preparation of 5,5″-Dichloro-2,2″-diethynyl-1,1′:4′,1″-terphenyl 26

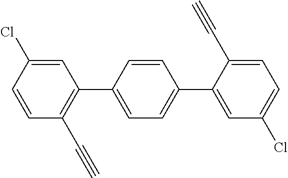 [SMILES: C#Cc1ccc(Cl)cc1-c1ccc(-c2cc(Cl)ccc2C#C)cc1]

2.00 g (3.64 mmol) of 24 were mixed with 80.0 mg (0.44 mmol) of copper(II) iodide and 30.0 ml of triethylamine and 10.0 ml of toluene. After degassing by argon bubbling, 160 mg (0.24 mmol) of bis(triphenylphosphine)palladium(II) dichloride and 1.50 ml (7.56 mmol) of (trimethylsilyl)acetylene were added. The reaction mixture was stirred at room temperature for 24 h under an inert atmosphere and monitored by thin-layer chromatography. The reaction mixture was filtered over a silica pad (DCM) to remove inorganic residues. The product thus obtained (1.52 g, 3.09 mmol, 85%) was then dissolved in a mixture of 100.0 ml THF and 100.0 ml methanol. Then, 3.00 g (21.74 mmol) potassium carbonate was added and the reaction mixture was stirred at room temperature for 24 h. The crude product was purified by column chromatography (hexane/ethyl acetate=9/1) to yield 0.73 g (2.10 mmol) of 26 in 68%.

¹H NMR (300 MHz, CD₂Cl₂): δ 7.67 (s, 4H), 7.58 (d, J=8.3, 2H), 7.46 (d, J=2.2, 2H), 7.33 (dd, J=2.2, 8.3, 2H), 3.19 (s, 2H).

¹³C NMR (75 MHz, CD₂Cl₂): δ 145.84, 139.27, 135.76, 135.48, 130.21, 129.51, 127.99, 119.56, 82.49, 81.78.

MS (FD, 8kV): m/z (%)=345.5 (100.0%, M⁺), (calc. C₂₂H₁₂Cl₂=347.24 g/mol).

Elemental Analysis: found 75.90% C, 4.08% H—calc. 76.10% C, 3.48% H.

### Example 2D Preparation of 4′,5′″-Dichloro-2,2″″,5,5″″-tetraphenyl-3,3″″,4,4″″-tetra(4-dodecylphenyl)-1,1′:2′,1″:4″,1″:2′″,1″″-quinquephenyl 28

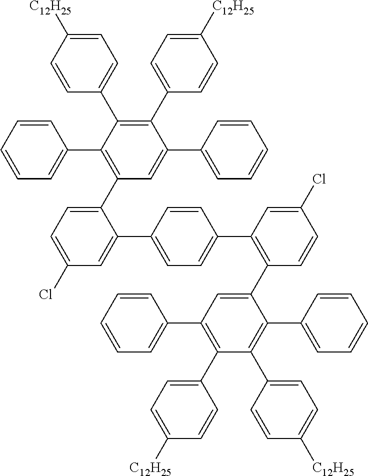

0.14 g (0.40 mmol) 26 and 0.70 g (0.97 mmol) 27 were placed in a microwave vessel. Then, 8.0 ml of ortho-xylene were added and the reaction mixture was degassed by argon bubbling. The reaction vessel was sealed, placed in a microwave reactor and heated to 160° C. at 300 W for 24 h with activated cooling. The crude product was pre-purified by column chromatography (hexane/ethyl acetate=9/1). Further purification was achieved by preparative gel permeation chromatography (chloroform) to yield 0.51 g (0.29 mmol) of 28 in 74% as a transparent oil which solidified upon standing.

¹H NMR (700 MHz, THF): δ 7.42 (d, J=4.9, 3H), 7.35 (d, J=8.1, 1H), 7.32-7.23 (m, 2H), 7.22 (s, 2H), 7.08 (t, J=10.6, 10H), 6.91 (d, J=53.1, 7H), 6.82 (s, 3H), 6.69 (s, 9H), 6.55 (m, 10H), 6.11 (s, 1H), 2.40 (t, J=7.5, 4H), 2.32 (t, J=7.1, 4H), 1.47 (p, 4H), 1.39 (p, 4H), 1.35-1.03 (m, 72H), 0.91 (t, J=6.9, 12H).

¹³C NMR (176 MHz, THF): δ 144.06, 143.93, 143.71, 142.36, 142.28, 141.72, 141.64, 141.43, 141.35, 141.31, 141.17, 141.06, 140.57, 139.44, 139.22, 135.19, 135.09, 134.48, 134.24, 134.03, 133.20, 132.77, 131.59, 131.18, 130.81, 129.13, 128.54, 128.32, 127.77, 126.97, 37.07, 33.78, 33.09, 31.59, 31.56, 31.37, 31.28, 30.74, 24.47, 15.37.

MS (FD, 8kV): m/z (%)=1730.9 (100.0%, M⁺), (calc. C₁₂₆H₁₄₈Cl₂=1733.43 g/mol).

Elemental Analysis: found 84.91% C, 8.95% H—calc. 87.30% C, 8.61% H (see general remarks “7.2.4 Elemental Combustion Analysis”).

### Example 3 Preparation of Polymer P2

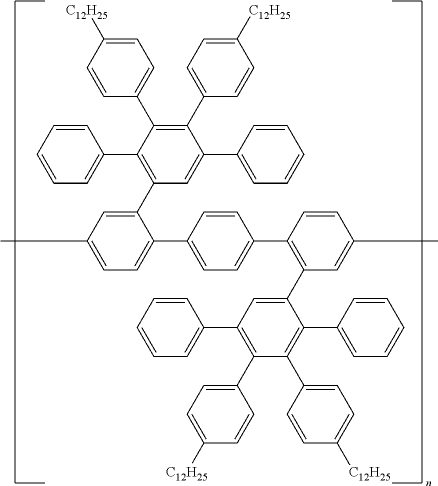

The catalyst solution was prepared inside the glove box by adding 0.5 ml DMF and 2.0 ml toluene to a mixture of 55.0 mg (0.19 mmol) bis(cyclooctadiene)nickel(0), 29.0 mg (0.19 mmol) 2,2′-bipyridine and 0.05 ml (0.19 mmol) cyclooctadiene. The resulting solution was stirred for 30 min at 60° C. Then, a solution of 100.0 mg (0.06 mmol) of 27 dissolved in 1.0 ml toluene and 0.5 ml DMF was added. The reaction mixture was stirred for 72 h at 80° C. under the exclusion of light. Then, excess chlorobenzene (anhydrous) was added and the mixture was stirred for additional 12 h. After cooling, the reaction mixture was slowly dropped into dilute methanolic hydrochloric acid. The white precipitate which formed was collected by filtration, re-dissolved in DCM and precipitated as described above for two more times to yield P2 as an off-white powder in 83%.

GPC: 76900 g/mol (PS).

FTIR: 3087 cm⁻¹, 3055 cm⁻¹, 3025 cm⁻¹, 2921 cm⁻¹, 1600 cm⁻¹, 1514 cm⁻¹, 1465 cm⁻¹, 1440 cm⁻¹, 1407 cm⁻¹, 1376 cm⁻¹, 1155 cm⁻¹, 1117 cm⁻¹, 1073 cm⁻¹, 1023 cm⁻¹, 1004 cm⁻¹, 839 cm⁻¹, 814 cm⁻¹, 757 cm⁻¹, 698 cm⁻¹, 614 cm⁻¹.

### Example 4 Preparation of Polymer P3

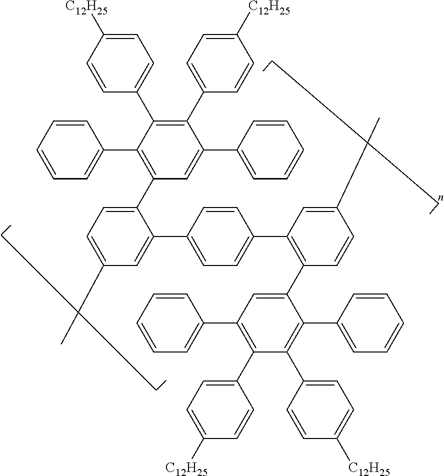

The catalyst solution was prepared inside the glove box by adding 0.5 ml DMF and 2.0 ml toluene to a mixture of 55.0 mg (0.19 mmol) bis(cyclooctadiene)nickel(0), 29.0 mg (0.19 mmol) 2,2′-bipyridine and 0.05 ml (0.19 mmol) cyclooctadiene. The resulting solution was stirred for 30 min at 60° C. Then, a solution of 100.0 mg (0.06 mmol) of 28 dissolved in 1.0 ml toluene and 0.5 ml DMF was added. The reaction mixture was stirred for 72 h at 80° C. under the exclusion of light. Then, excess chlorobenzene (anhydrous) was added and the mixture was stirred for additional 12 h. After cooling, the reaction mixture was slowly dropped into dilute methanolic hydrochloric acid. The white precipitate which formed was collected by filtration, re-dissolved in DCM and precipitated as described above for two more times to yield P3 as an off-white powder in 81%.

GPC: 11400 g/mol (PS).

FTIR: 3083 cm⁻¹, 3056 cm⁻¹, 3025 cm⁻¹, 2922 cm⁻¹, 2852 cm⁻¹, 1601 cm⁻¹, 1514 cm⁻¹, 1465 cm⁻¹, 1439 cm⁻¹, 1407 cm⁻¹, 1377 cm⁻¹, 1261 cm⁻¹, 1074 cm⁻¹, 1023 cm⁻¹, 1008 cm⁻¹, 896 cm⁻¹, 823 cm⁻¹, 801 cm⁻¹, 755 cm⁻¹, 721 cm⁻¹, 698 cm⁻¹, 655 cm⁻¹.

Initial analysis of P1 and P2 by MALDI-TOF spectroscopy indicated the presence of a regular pattern which extended up to molecular weights of 35000-40000 g/mol. The number of repeat units was between 20 and 24 for both polymers. Due to the rigid poly(para-phenylene) backbone, a length between 22 nm and 27 nm can be derived for the longest chains of the mixture.

FIG. 5 shows the MALDI-TOF spectra of P1 and P2 reflecting the power of the polymerization approach. In the case of P1 and P2 already the heptamer is composed of 546 regularly arranged aromatic carbon atoms and 91 benzene rings. A high number of carbon-carbon bonds are pre-formed upon synthesis of the polymeric precursors and prior to the actual cyclodehydrogenation step.

The Maximization of the molecular weight via the AA-type Yamamoto approach has thus been achieved.

### Example 5 Preparation of Graphene Nanoribbon GNR2

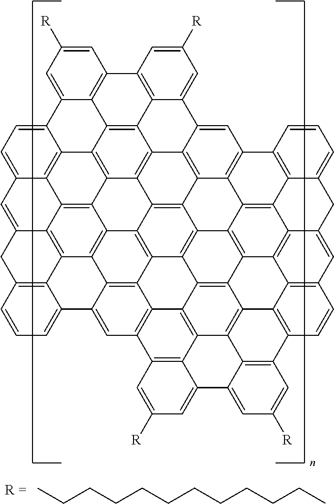

Method 1 (FeCl₃)

In a typical experiment, 25.0 mg of P2 was dissolved in 30.0 ml DCM. Then, 0.51 g (3.16 mmol, 7.5 eqv./H) ferric chloride, dissolved in 2.0 ml nitromethane were added. Through the reaction mixture was passed for 2 h a stream of argon saturated with DCM in order to prevent evaporation of the reaction solvent. The reaction was stirred at room temperature for 24 h. Then, excess methanol was added and the precipitate that formed was collected by filtration and washed with water and methanol. After drying, 23.0 mg of a black solid were obtained in 91%.

Method 2 (PIFA/BF₃)

In a typical experiment 25.0 mg of P2 was dissolved in 20.0 ml anhydrous DCM. Then, 200.0 mg phenyliodine(III) bis(trifluoroacetate (PIFA, 0.45 mmol, 2.1 eqv./bond) and 63.0 mg (0.056 ml, 0.45 mmol, 2.1 eqv./bond) boron trifluoride etherate dissolved in 2.0 ml anhydrous DCM were added at a temperature of −60° C. (chloroform/dry ice). The reaction was stirred under an inert atmosphere at this temperature for 2 h and at room temperature for additional 24 h. Then, excess methanol and water was added and the precipitate that formed was collected by filtration and washed with methanol. After drying, 24.0 mg of a black solid were obtained in 95%.

FTIR: 3063 cm⁻¹, 2920 cm⁻¹, 2849 cm⁻¹, 1718 cm⁻¹, 1603 cm⁻¹, 1587 cm⁻¹, 1452 cm⁻¹, 1302 cm⁻¹, 1215 cm⁻¹, 1076 cm⁻¹, 1012 cm⁻¹, 870 cm⁻¹, 818 cm⁻¹, 723 cm⁻¹, 620 cm⁻¹.

Raman: 1593 cm⁻¹, 1292 cm⁻¹.

### Example 6 Preparation of Graphene Nanoribbon GNR3

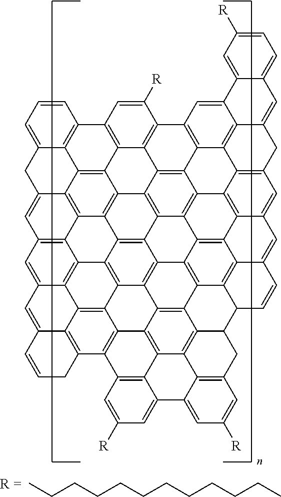

Method 1 (FeCl₃)

In a typical experiment, 25.0 mg of P3 was dissolved in 30.0 ml DCM. Then, 0.51 g (3.16 mmol, 7.5 eqv./H) ferric chloride, dissolved in 2.0 ml nitromethane were added. Through the reaction mixture was passed for 2 h a stream of argon saturated with DCM in order to prevent evaporation of the reaction solvent. The reaction was stirred at room temperature for 24 h. Then, excess methanol was added and the precipitate that formed was collected by filtration and washed with water and methanol. After drying, 23.5 mg of a black solid were obtained in 92%.

Method 2 (PIFA/BF₃)

In a typical experiment 25.0 mg of P3 was dissolved in 20.0 ml anhydrous DCM. Then, 200.0 mg phenyliodine(III) bis(trifluoroacetate (PIFA, 0.45 mmol, 2.1 eqv./bond) and 63.0 mg (0.056 ml, 0.45 mmol, 2.5 eqv./bond) boron trifluoride etherate dissolved in 2.0 ml anhydrous DCM were added at a temperature of −60° C. (chloroform/dry ice). The reaction was stirred under an inert atmosphere at this temperature for 2 h and at room temperature for additional 24 h. Then, excess methanol and water was added and the precipitate that formed was collected by filtration and washed with methanol. After drying, 20.0 mg of a black solid were obtained in 85%.

FTIR: 3065 cm⁻¹, 2919 cm⁻¹, 2850 cm⁻¹, 1724 cm⁻¹, 1604 cm⁻¹, 1582 cm⁻¹, 1452 cm⁻¹, 1367 cm⁻¹, 1337 cm⁻¹, 1305 cm⁻¹, 1208 cm⁻¹, 1150 cm⁻¹, 1078 cm⁻¹, 861 cm⁻¹, 822 cm⁻¹, 760 cm⁻¹, 718 cm⁻¹, 624 cm⁻¹.

Raman: 1583 cm⁻¹, 1294 cm⁻¹.

The Raman spectrum of GNR2 is shown in FIG. 6

### Example 7 Preparation of 2,2′-(4,4″-Dichloro-[1,1′:4′,1″-terphenyl]-2,2″-diyl)bis(1,4-diphenyltriphenylene) 29

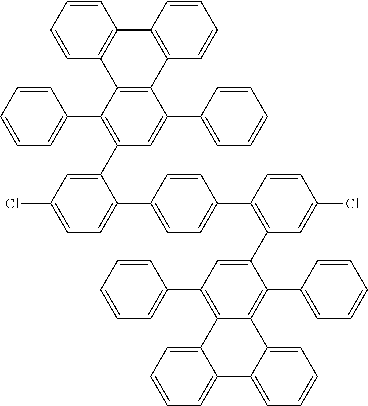 [SMILES: Clc1ccc(-c2ccc(-c3ccc(Cl)cc3-c3cc(-c4ccccc4)c4c5ccccc5c5ccccc5c4c3-c3ccccc3)cc2)c(-c2cc(-c3ccccc3)c3c4ccccc4c4ccccc4c3c2-c2ccccc2)c1]

0.15 g (0.43 mmol) 21 and 0.50 g (1.30 mmol) phencyclone were placed in a microwave vessel. Then, 8.0 ml of ortho-xylene were added and the reaction mixture was degassed by argon bubbling. The reaction vessel was sealed, placed in a microwave reactor and heated to 160° C. at 300 W for 24 h with activated cooling. The crude product was pre-purified by column chromatography (hexane/ethyl acetate=9/1). Further purification was achieved by preparative gel permeation chromatography (chloroform) to yield 0.27 g (0.26 mmol) of 29 in 76% as a colorless solid.

¹H NMR (700 MHz, THF) δ 8.45 (dd, J=7.9, 25.4, 1H), 8.37 (dd, J=7.9, 42.3, 3H), 7.89 (s, 1H), 7.74 (dd, J=8.1, 41.0, 2H), 7.54 (s, 2H), 7.53-7.48 (m, 3H), 7.48-7.22 (m, 14H), 7.19 (dd, J=2.3, 8.5, 2H), 7.17 (d, J=8.2, 2H), 7.12 (dt, J=4.7, 12.0, 2H), 7.04 (t, J=7.2, 1H), 7.02-6.91 (m, 4H), 6.89 (d, J=8.5, 2H), 6.82 (m, 3H), 6.70 (t, J=7.2, 1H), 6.32 (d, J=383.1, 1H), 6.38 (s, 1H), 6.22 (s, 1H), 5.99 (d, J=413.2, 2H).

¹³C NMR (75 MHz, CD₂Cl₂): δ 145.61, 145.50, 142.99, 142.69, 142.31, 142.04, 140.18, 139.72, 139.19, 137.79, 137.71, 134.32, 134.21, 133.37, 133.09, 132.89, 132.48, 132.37, 132.25, 132.03, 131.74, 131.43, 130.98, 130.81, 130.01, 129.25, 128.10, 127.70, 127.31, 127.11, 126.87, 126.32, 126.07, 125.90, 124.35, 124.16, 124.06.

MS (FD, 8kV): m/z (%)=1053.9 (100.0%, M⁺), (calc. C₇₈H₄₈Cl₂=1056.12 g/mol).

Elemental Analysis: found 85.07% C, 4.88% H—calc. 88.71% C, 4.58% H (see general remarks “7.2.4 Elemental Combustion Analysis”).

### Example 8 Preparation of 2,2′-(5,5″-Dichloro-[1,1′:4′,1″-terphenyl]-2,2″-diyl)bis(1,4-diphenyltriphenylene) 30

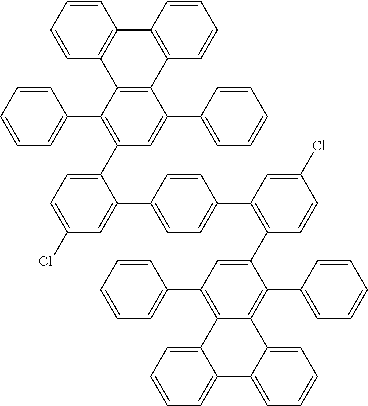 [SMILES: Clc1ccc(-c2cc(-c3ccccc3)c3c4ccccc4c4ccccc4c3c2-c2ccccc2)c(-c2ccc(-c3cc(Cl)ccc3-c3cc(-c4ccccc4)c4c5ccccc5c5ccccc5c4c3-c3ccccc3)cc2)c1]

0.20 g (0.58 mmol) 26 and 0.55 g (1.44 mmol) phencyclone were placed in a microwave vessel. Then, 8.0 ml of ortho-xylene were added and the reaction mixture was degassed by argon bubbling. The reaction vessel was sealed, placed in a microwave reactor and heated to 160° C. at 300 W for 24 h with activated cooling. The crude product was pre-purified by column chromatography (hexane/ethyl acetate=9/1). Further purification was achieved by preparative gel permeation chromatography (chloroform) to yield 0.52 g (0.49 mmol) of 30 in 85% as a colorless solid.

¹H NMR (500 MHz, THF) δ 8.44 (dd, J=8.0, 12.8, 1H), 8.40 (d, J=7.9, 1H), 8.34 (d, J=7.8, 1H), 7.88 (s, 1H), 7.71 (dd, J=8.3, 40.1, 2H), 7.50 (s, 2H), 7.46-7.21 (m, 18H), 7.21-7.15 (m, 2H), 7.10 (t, J=7.7, 2H), 7.05-6.95 (m, 3H), 6.93 (dd, J=2.1, 11.3, 3H), 6.86 (t, J=7.4, 2H), 6.70 (t, J=7.8, 2H), 6.55 (s, 1H), 6.30 (s, 4H), 5.74 (s, 1H).

¹³C NMR (126 MHz, THF) δ 146.72, 144.43, 143.69, 143.24, 140.88, 140.18, 138.88, 136.20, 136.05, 135.89, 134.93, 134.78, 134.59, 134.22, 134.00, 133.57, 132.77, 132.47, 132.12, 131.70, 131.32, 131.17, 131.03, 130.65, 130.42, 129.75, 129.34, 129.01, 128.64, 128.03, 127.63, 127.36, 126.74, 126.35, 126.03, 125.75, 124.78, 124.50.

MS (FD, 8kV): m/z (%)=1054.8 (100.0%, M⁺), (calc. C₇₈H₄₈Cl₂=1056.12 g/mol).

Elemental Analysis: found 85.53% C, 5.59% H—calc. 88.71% C, 4.58% H (see general remarks “7.2.4 Elemental Combustion Analysis”).

### Example 9A Preparation of 4,4″-Dibromo-[1,1′:4′,1″-terphenyl]-2,2″-diamine 32

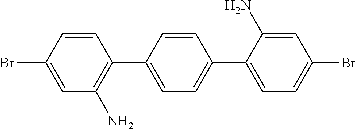 [SMILES: Nc1cc(Br)ccc1-c1ccc(-c2ccc(Br)cc2N)cc1]

1.47 g (3.08 mmol) 31 and 0.20 g of palladium on carbon (10 wt %) were suspended in 50.0 ml of THF. The reaction mixture was evacuated after what a balloon filled with hydrogen gas was connected. The reaction mixture was heated to 50° C. for 24 h under vigorous stirring and monitored by thin-layer chromatography. With the consumption of the starting compound the reaction mixture turned homogenous. The crude product was purified by filtration to yield 1.21 g (2.89 mmol) of 32 as an orange solid in 94%.

¹H NMR (300 MHz, CD₂Cl₂): δ 7.51 (s, 4H), 7.19 (tt, J=7.1, 13.9, 4H), 6.95 (m, 2H), 4.03 (s, 4H).

¹³C NMR (75 MHz, CD₂Cl₂): δ 145.87, 138.29, 132.27, 130.02, 126.44, 122.58, 121.80, 118.53.

MS (FD, 8kV): m/z (%)=417.8 (100.0%, M⁺), (calc. C₁₈H₁₄Br₂N₂=418.13 g/mol).

### Example 9B Preparation of 4,4″-Dibromo-2,2″-diiodo-1,1′:4′,1″-terphenyl 33

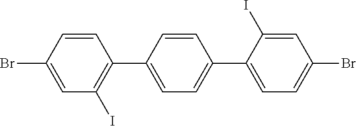 [SMILES: Brc1ccc(-c2ccc(-c3ccc(Br)cc3I)cc2)c(I)c1]

1.20 g (2.85 mmol) of 32 was suspended in 7.0 ml of water. Then, 4.0 ml of concentrated hydrochloric acid were added under cooling. At a temperature of −5° C., 4.0 ml of an aqueous solution containing 0.50 g (7.06 mmol) sodium nitrite were added dropwise. During this procedure, the color of the reaction mixture changed from yellow to dark brown. Subsequently, 12.0 ml of an aqueous solution containing 5.00 g (28.52 mmol) potassium iodide were added dropwise while maintaining the temperature below 0° C. After the addition, the reaction was allowed to proceed for 1 h at room temperature. After extraction with DCM, treatment with an aqueous solution of sodium thiosulfate and removal of the solvent under reduced pressure the crude product was purified by column chromatography (hexane/ethyl acetate=8/2) to yield 0.77 g (1.20 mmol) of 33 in 42% as an orange solid.

¹H NMR (300 MHz, CD₂Cl₂): δ 8.15 (d, J=2.0, 2H), 7.57 (dd, J=2.0, 8.2, 2H), 7.39 (s, 4H), 7.25 (d, J=8.2, 2H).

¹³C NMR (75 MHz, CD₂Cl₂): δ 145.72, 143.22, 142.06, 131.96, 131.62, 129.48, 122.19, 99.27.

MS (FD, 8kV): m/z (%)=639.9 (100.0%, M⁺), (calc. C₁₈H₁₀Br₂I₂=639.89 g/mol).

### Example 9C Preparation of 4,4″-Dibromo-2,2″-diethynyl-1,1′:4′,1″-terphenyl 35

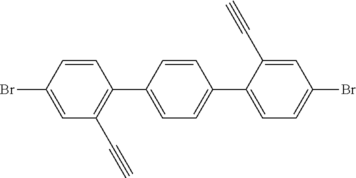 [SMILES: C#Cc1cc(Br)ccc1-c1ccc(-c2ccc(Br)cc2C#C)cc1]

0.60 g (0.99 mmol) of 33 was mixed with 25.0 mg (0.14 mmol) of copper(II) iodide and 10.0 ml of triethylamine. After degassing by argon bubbling, 50 mg (0.08 mmol) of bis(triphenylphosphine)palladium(II) dichloride and 0.40 ml (2.01 mmol) of (trimethylsilyl)acetylene were added. The reaction mixture was stirred at room temperature for 24 h under an inert atmosphere and monitored by thin-layer chromatography. The reaction mixture was filtered over a silica pad (DCM) to remove inorganic residues.

The product thus obtained (0.41 g, 0.71 mmol, 72%) was then dissolved in a mixture of 20.0 ml THF and 20.0 ml methanol. Then, 0.55 g (3.95 mmol) potassium carbonate was added and the reaction mixture was stirred at room temperature for 24 h. The crude product was purified by column chromatography (hexane/ethyl acetate=9/1) to yield 0.19 g (0.43 mmol) of 35 in 60%.

¹H NMR (300 MHz, CD₂Cl₂): δ 7.79 (d, J=2.1, 2H), 7.65 (s, 4H), 7.58 (dd, J=2.1, 8.4, 2H), 7.33 (d, J=8.4, 2H), 3.19 (s, 2H).

¹³C NMR (75 MHz, CD₂Cl₂): δ 143.28, 139.27, 136.96, 132.90, 131.70, 129.46, 122.86, 121.35, 82.11, 68.34.

MS (FD, 8kV): m/z (%)=436.0 (100.0%, M⁺), (calc. C₂₂H₁₂Br₂=436.14 g/mol).

Elemental Analysis: found 68.12% C, 6.60% H—calc. 60.59% C, 2.77% H.

### Example 9D Preparation of 2,2′-(4,4″-Dibromo-[1,1′:4′,1″-terphenyl]-2,2″-diyl)bis(1,4-diphenyltriphenylene) 36

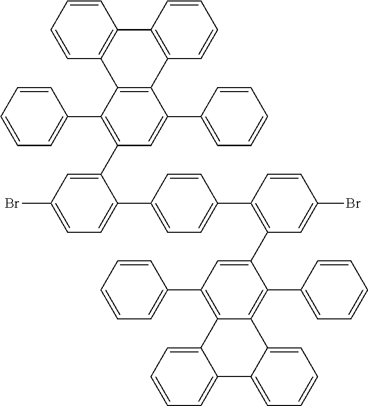 [SMILES: Brc1ccc(-c2ccc(-c3ccc(Br)cc3-c3cc(-c4ccccc4)c4c5ccccc5c5ccccc5c4c3-c3ccccc3)cc2)c(-c2cc(-c3ccccc3)c3c4ccccc4c4ccccc4c3c2-c2ccccc2)c1]

0.15 g (0.34 mmol) 35 and 0.33 g (0.86 mmol) phencyclone were placed in a microwave vessel. Then, 3.0 ml of ortho-xylene were added and the reaction mixture was degassed by argon bubbling. The reaction vessel was sealed, placed in a microwave reactor and heated to 160° C. at 300 W for 24 h with activated cooling. The crude product was pre-purified by column chromatography (hexane/ethyl acetate=9/1). Further purification was achieved by preparative gel permeation chromatography (chloroform) to yield 15 mg (0.31 mmol) of 36 in 90% as an off-white solid.

¹H-NMR (700 MHz, THF): δ 8.45 (dd, J=8.0, 25.6, 1H), 8.37 (dd, J=7.9, 42.2, 2H), 7.89 (s, 1H), 7.74 (dd, J=8.1, 41.1, 2H), 7.66 (d, J=2.1, 1H), 7.54 (d, J=3.0, 2H), 7.49 (s, 1H), 7.43 (dt, J=7.6, 15.9, 3H), 7.38-7.29 (m, 10H), 7.27 (dd, J=5.0, 13.1, 2H), 7.16 (d, J=8.3, 2H), 7.12 (t, J=7.7, 2H), 7.04 (t, J=7.2, 1H), 7.02-6.90 (m, 4H), 6.83 (t, J=7.1, 4H), 6.75 (d, J=8.5, 1H), 6.70 (t, J=7.7, 1H), 6.37 (s, 1H), 6.24 (s, 1H), 6.22 (s, 4H), 6.09-5.99 (m, 1H), 5.65 (s, 1H).

¹³C-NMR (176 MHz, THF): δ 145.65, 145.55, 143.34, 143.03, 142.33, 142.07, 140.85, 140.64, 139.68, 139.33, 139.24, 137.83, 137.75, 135.66, 135.31, 134.39, 134.28, 132.92, 132.69, 132.60, 132.53, 132.32, 131.22, 131.03, 130.96, 130.82, 129.25, 128.16, 127.76, 127.36, 126.92, 126.44, 126.37, 126.05, 125.95, 124.41, 124.22, 124.12, 121.49.

MS (MALDI-TOF): m/z (%)=1144.23 (100.0%), 1145.35 (87.4%), 1146.25 (77.9%), 1147.20 (49.8%), 1143.28 (40.9%), 1142.24 (40.5%), 1148.15 (20.73%), (calc. C₇₈H₄₈Br₂=1145.02 g/mol—isotop. distr.: 1144.21 (100.0%), 1145.21 (84.4%), 1142.21 (51.4%), 1146.21 (48.6%), 1143.22 (43.6%), 1147.21 (41.3%), 1146.22 (35.6%)).

Elemental Analysis: found 87.37% C, 4.03% H—calc. 81.82% C, 4.23% H (see general remarks “7.2.4 Elemental Combustion Analysis”).

The molecular weight of this compound (M=1145.02 g/mol) is still higher than in the previous two cases. UHV sublimation of this large oligophenylene can be realized at a temperature of 380° C. The STM results obtained from monomer 36 suggests the successful formation of laterally extended GNR.

### Example 9E

A chemistry-driven protocol for the direct growth of GNRs and graphene networks on surfaces has been very recently established (see Cai, J.; et al. Nature 466, 470-473 (2010).

In analogy, the molecular precursor 2,2′-(4,4″-Dibromo-[1,1′:4′,1″-terphenyl]-2,2″-diyl)bis(1,4-diphenyltriphenylene) 36 was sublimated at a rate of 1 Å/min for 100 seconds onto a clean Au(111) single crystal substrate which was cleaned by repeated cycles of argon ion bombardment and annealing to 480° C. The substrate was maintained at room temperature during deposition and then immediately heated to 500° C. to induce diradical formation, polymerization. Then the sample was post-annealed at the same temperature for 5 min to cyclodehydrogenate the polymers. As it can be seen from the STM image in FIG. 7, the metal substrate is densely covered with ribbon-type structures that formed from monomer 36 and reach maximum lengths of 30 nm to 40 nm. For the polymerization and cyclodehydrogenation the pathway is schematically depicted in FIG. 8.

Comparison of the length of the surface-bound GNR structures suggests that the polymerization proceeded to a higher degree in the case of bromine-functionalized 36 as compared to chlorine-functionalized monomers 29 and 30.

1. An oligophenylene monomer of formulae A, B, C, D, E or F 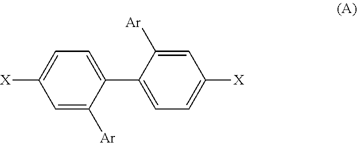
wherein
Ar is 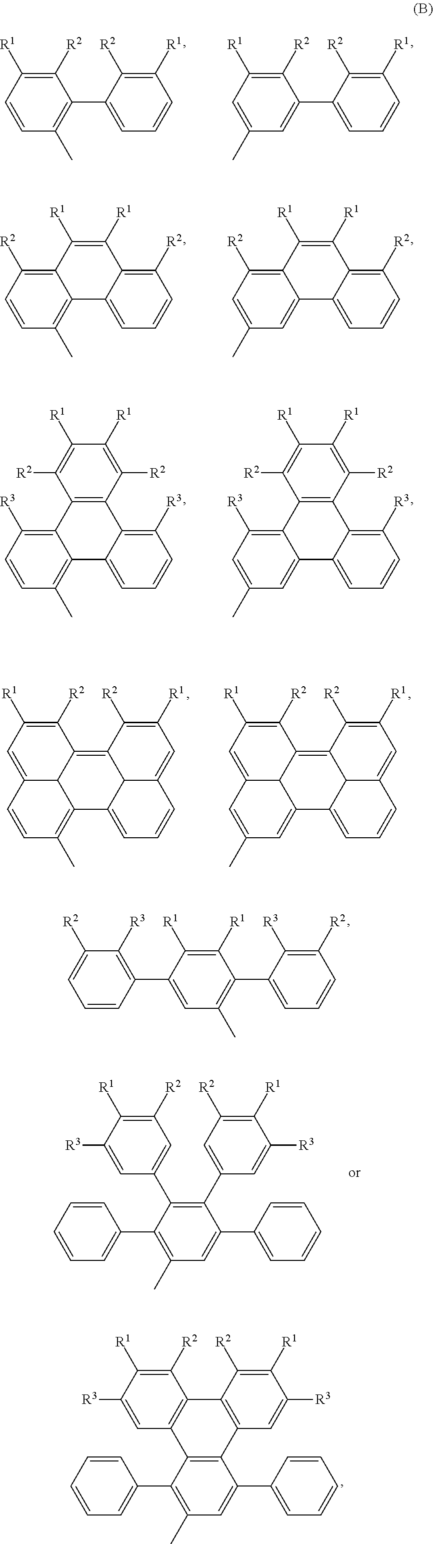 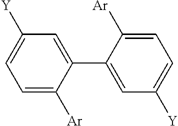
wherein
Ar is 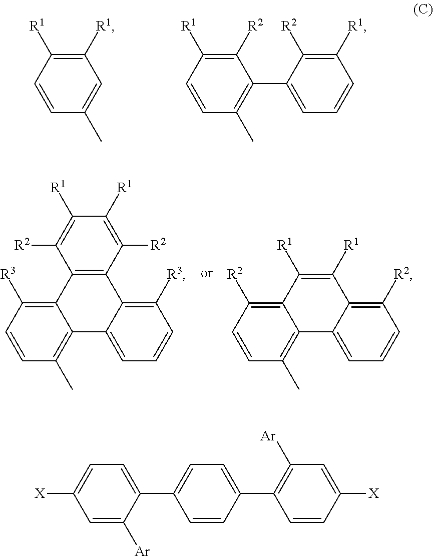
wherein
Ar is 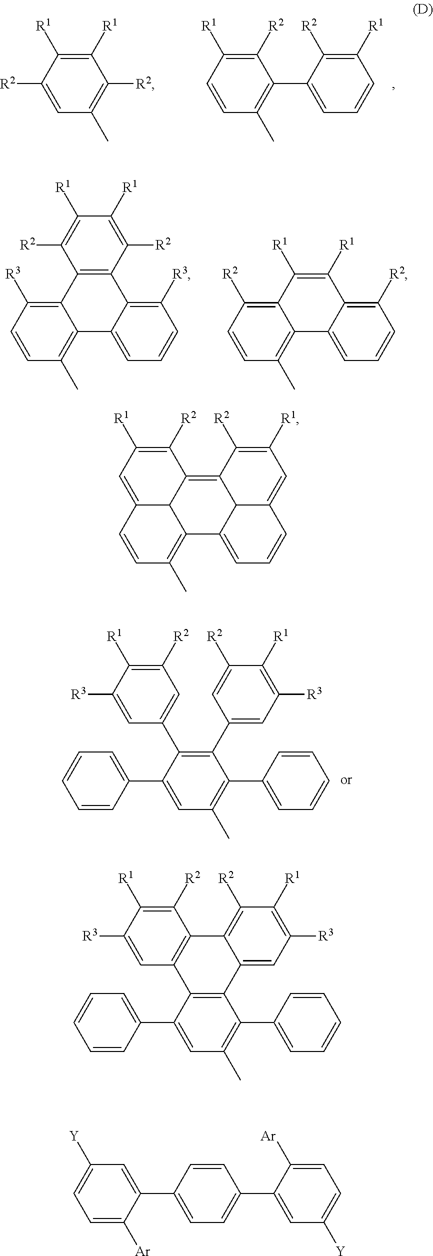
wherein
Ar is 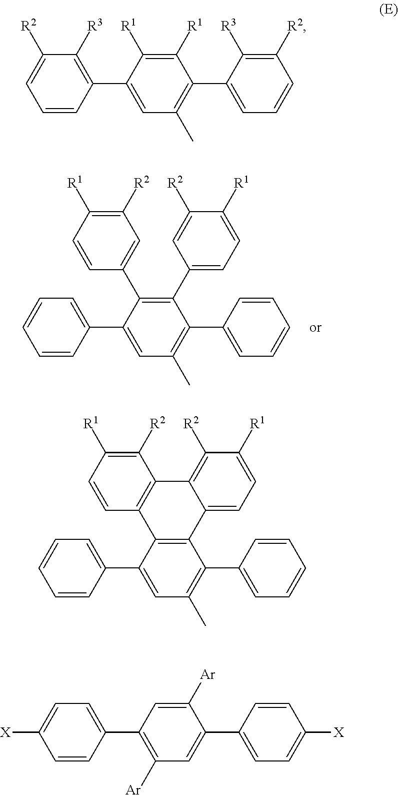
wherein
Ar is 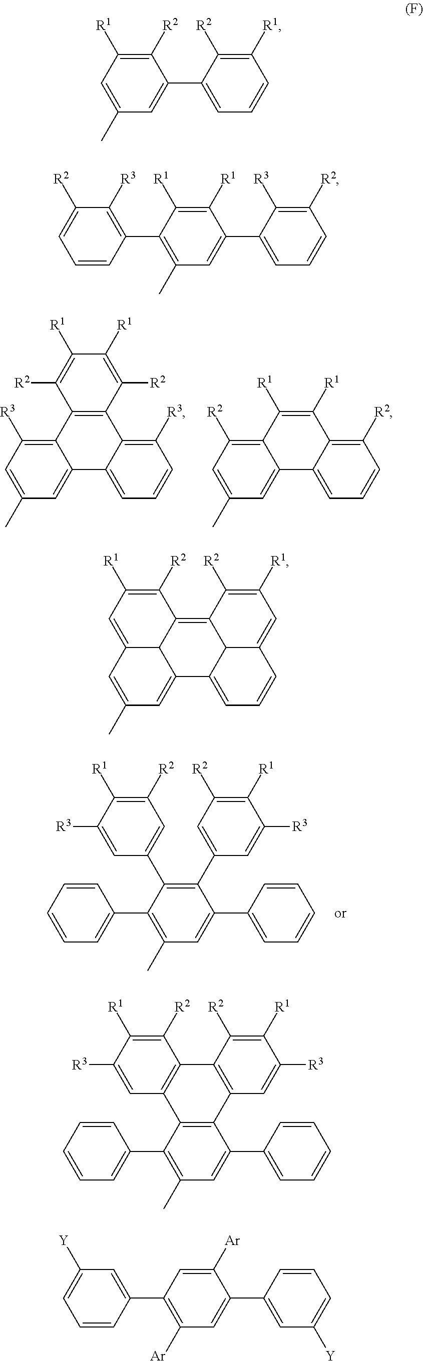
wherein
Ar is 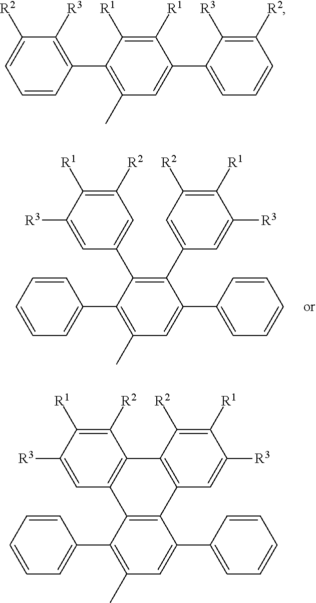
wherein, in each of formulae A, B, C, D, E and F,
X and Y are halogen, trifluoromethylsulfonate or diazonium
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue.

2. The oligophenylene monomer of claim 1, having a formula selected from the group consisting of formulae I, II, III and IV 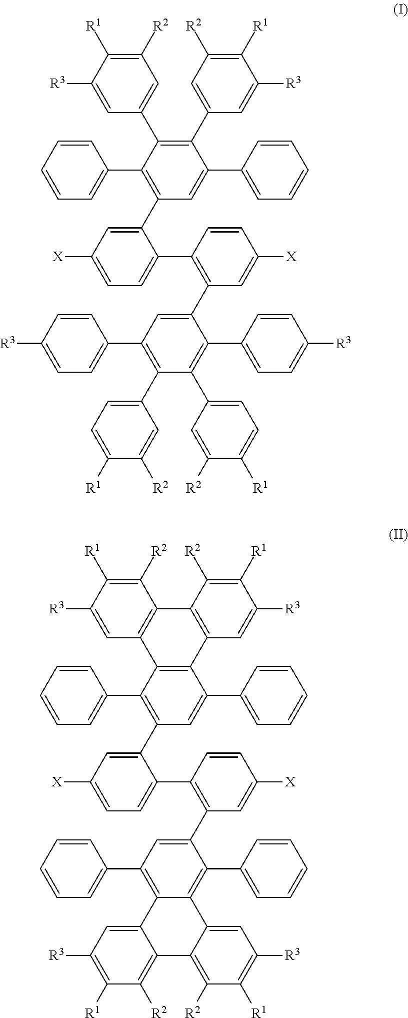
wherein
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue,
X=halogen, 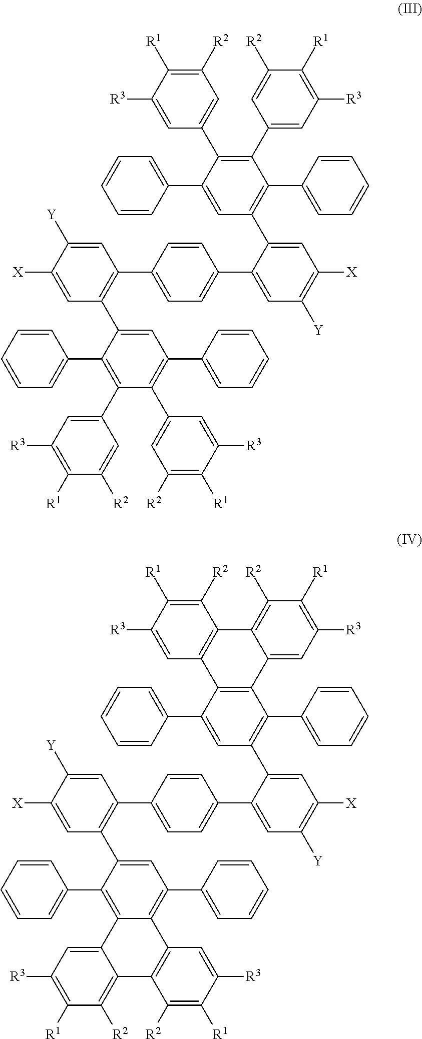
wherein
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue,
X=halogen and Y═H, or X═H and Y=halogen,
with the proviso that R³═H if X═H and Y=halogen.

3. The oligophenylene monomer of claim 2, wherein X and Y are each independently Cl or Br.

4. A polymeric precursor suitable for the preparation of a graphene nanoribbon, obtained from the oligophenylene monomer of claim 1.

5. The polymeric precursor of claim 4 having a repeating unit of formulae V, VI, VII, VIII, IX or X, 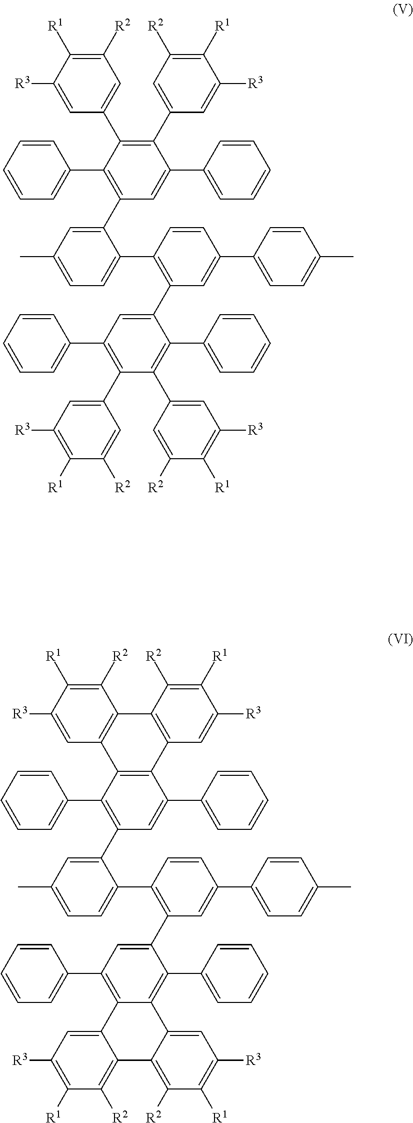 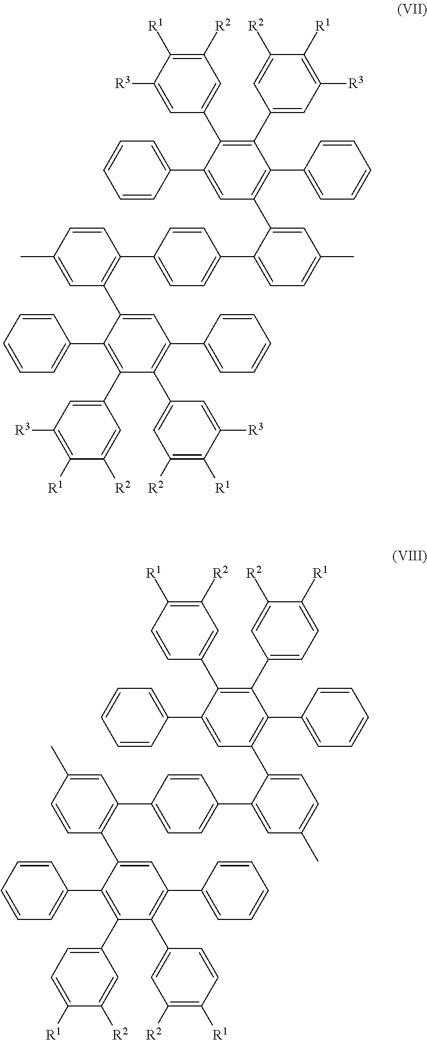 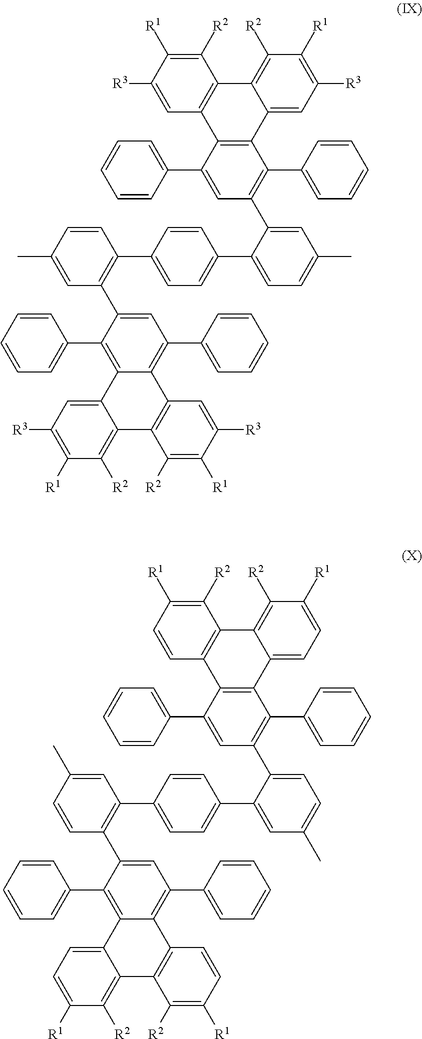
wherein R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue.

6. The polymeric precursor of claim 5, having the formula V, obtained by copolymerization of an oligophenylene monomer of formula I 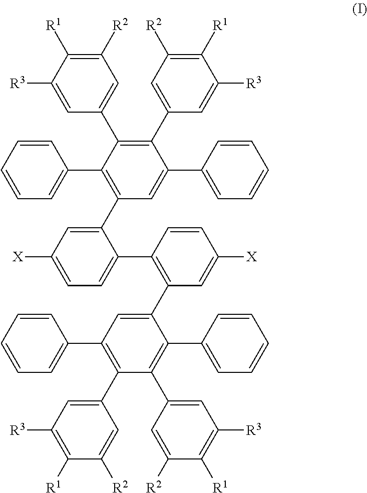
wherein
R¹, R², R³ are each independently H, halogen, —OH, —NH₂, —CN, —NO₂, a linear or branched, saturated or unsaturated hydrocarbon residue, optionally substituted 1- to 5-fold with halogen, —OH, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue,
X=halogen,
with 1,4-phenyldiboronic acid or 1,4-phenyldiboronic acid ester.

7. The polymeric precursor of claim 5, having the formula VI, obtained by copolymerization of an oligophenylene monomer of formula II 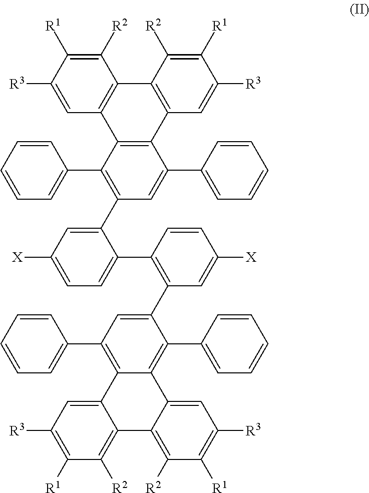
wherein
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue,
X=halogen
with 1,4-phenyldiboronic acid or 1,4-phenyldiboronic acid ester.

8. The polymeric precursor of claim 5, having the formula VII, obtained by Yamamoto-polymerization of a monomer of formula IIIa 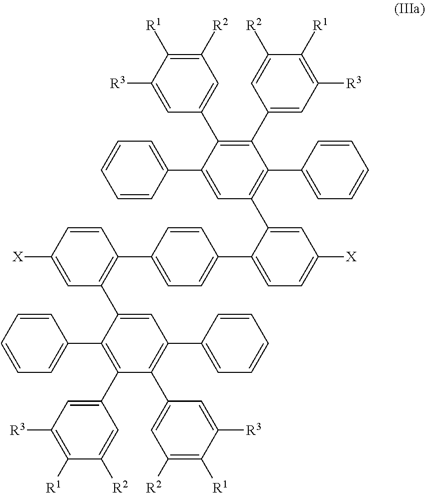
wherein
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue,
X is halogen, trifluoromethylsulfonate or diazonium.

9. The polymeric precursor of claim 5, having the formula VIII, obtained by Yamamoto-polymerization of a monomer of formula IIIb 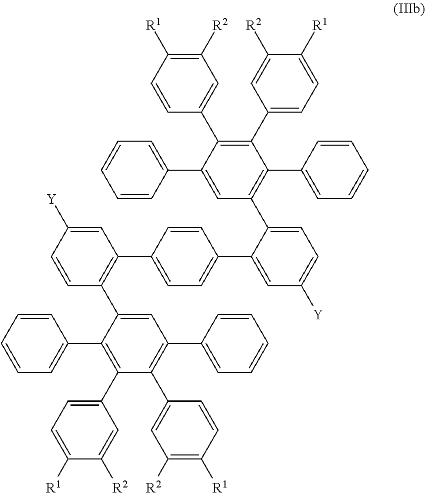
wherein
R¹, R² are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue,
Y=halogen, trifluoromethylsulfonate or diazonium.

10. The polymeric precursor of claim 5, having the formula X, obtained by Yamamoto-polymerization of a monomer of formula IVa 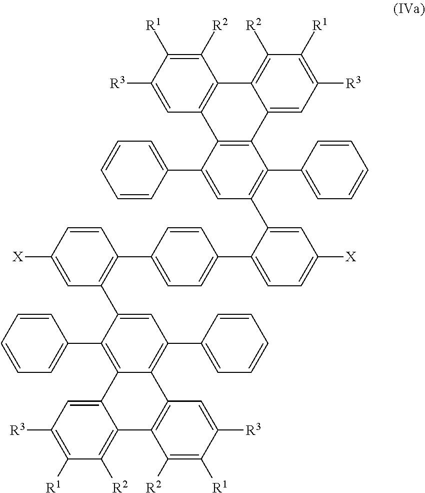
wherein
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue,
X=halogen, trifluoromethylsulfonate or diazonium.

11. The polymeric precursor of claim 5, having the formula X, obtained by Yamamoto-polymerization of a monomer of formula IVb 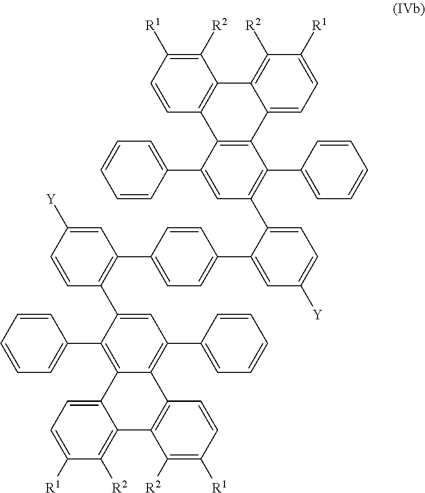
wherein
R¹, R² are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue,
Y=halogen, trifluoromethylsulfonate or diazonium.

12. A graphene nanoribbon obtained by cyclodehydrogenation of the polymeric precursor of claim 5.

13. The graphene nanoribbon of claim 12, prepared by a solution process.

14. The graphene nanoribbon of claim 12, prepared by direct growth of the graphene nanoribbon on a surface by polymerization and cyclodehydrogenation.

15. The graphene nanoribbon of claim 14, obtained from a monomer of formula IV 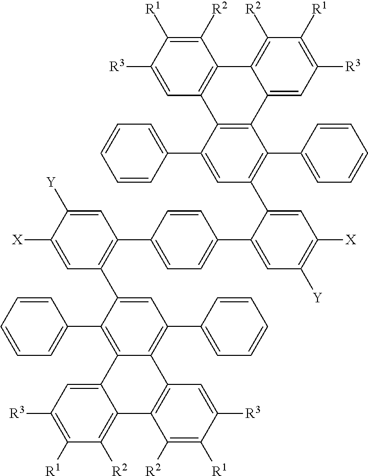
wherein
X, Y is halogen, trifluoromethylsulfonate or diazonium
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue with the proviso that either both X or both Y are hydrogen,
by direct growth of the graphene nanoribbon on a surface by polymerization of the monomer and cyclodehydrogenation.

16. A process for the preparation of an oligophenylene monomer of formula I 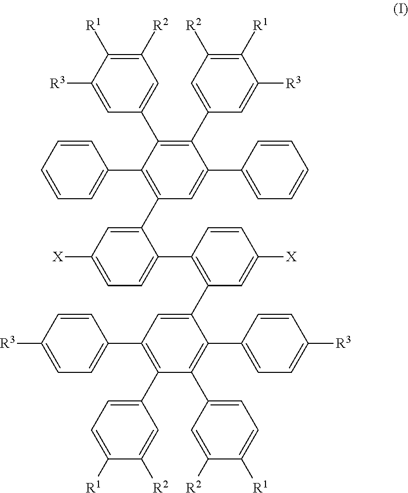
by Diels-Alder reaction of 4,4′-dibromo-2,2′-diethynyl-1,1′-biphenyl 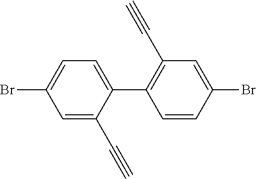 [SMILES: C#Cc1cc(Br)ccc1-c1ccc(Br)cc1C#C]
with tetraphenylcyclopentadienone 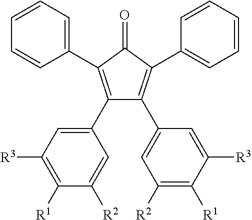
wherein
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue.

17. A process for the preparation of an oligophenylene monomer of formula II 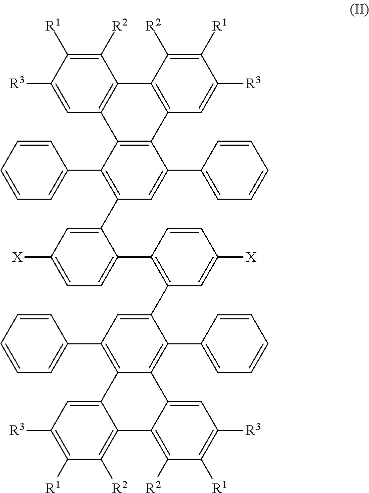
by Diels-Alder reaction of 4,4′-dibromo-2,2′-diethynyl-1,1′-biphenyl 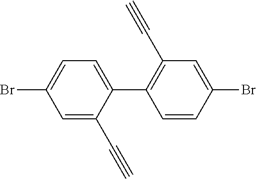 [SMILES: C#Cc1cc(Br)ccc1-c1ccc(Br)cc1C#C]
with phencyclone 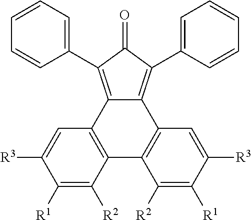
wherein
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue.

18. A process for the preparation of a monomer of formula IIIa 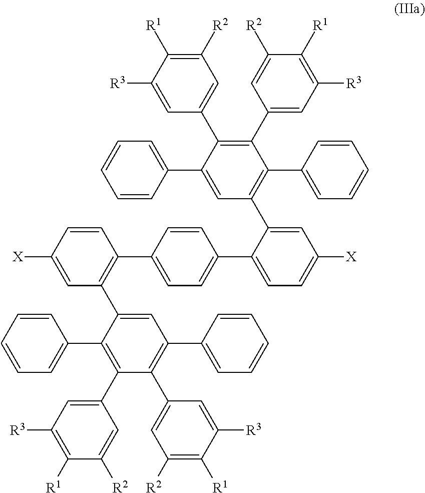
wherein X=halogen
by Diels-Alder reaction of 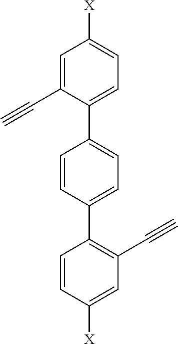
wherein X is halogen,
with tetraphenylcyclopentadienone 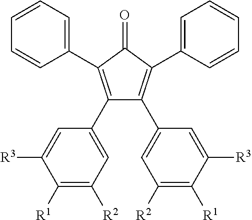
wherein
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue.

19. A process for the preparation of a monomer of formula IIIb 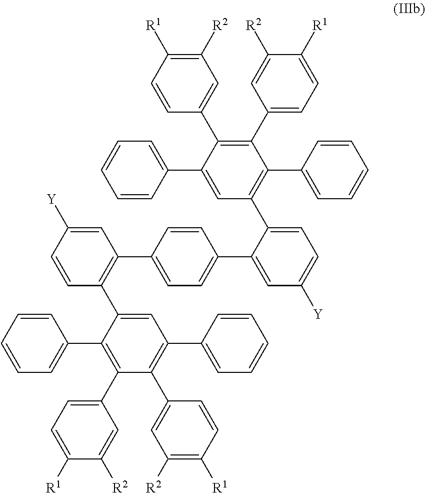
wherein Y=halogen,
by Diels-Alder reaction of 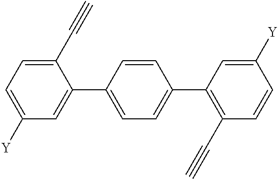
wherein Y=halogen,
with tetraphenylcyclopentadienone 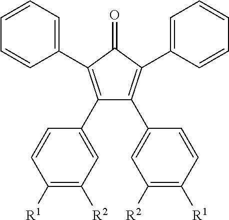
wherein
R¹, R² are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue.

20. A process for the preparation of a monomer of formula IVa 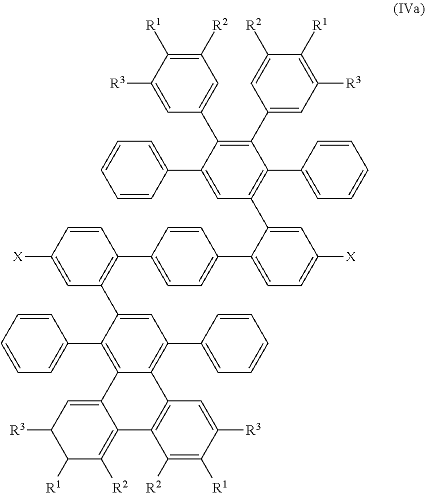
wherein X=halogen,
by Diels-Alder reaction of 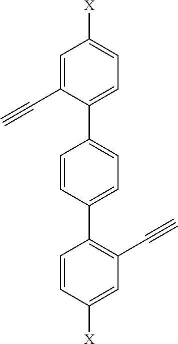
wherein X=halogen,
with phencyclone 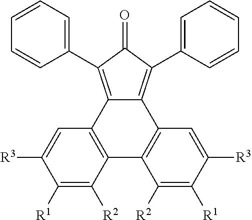
wherein
R¹, R², R³ are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue.

21. A process for the preparation of a monomer of formula IVb 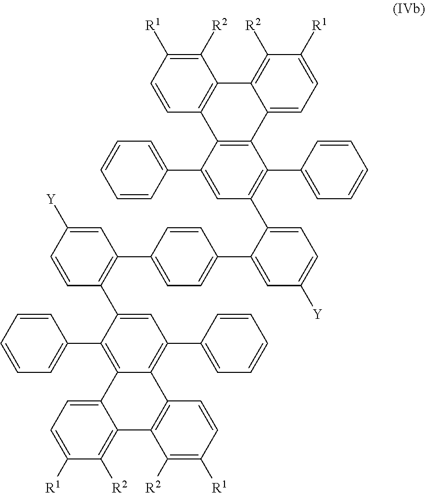
wherein Y=halogen,
by Diels-Alder reaction of 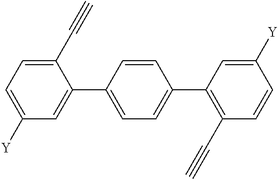
wherein Y=halogen,
with phencyclone 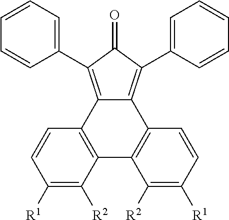
wherein
R¹, R² are each independently H, halogen, —OH, —NH₂, —CN, —NO₂, a linear or branched, saturated or unsaturated C₁-C₄₀ hydrocarbon residue, optionally substituted 1- to 5-fold with halogen, —OH, —NH₂, —CN and/or —NO₂, and wherein one or more CH₂-groups can be replaced by —O—, —S—, —C(O)O—, —O—C(O)—, —C(O)—, —NH— or —NR—, wherein R is an optionally substituted C₁-C₄₀ hydrocarbon residue, or an optionally substituted aryl, alkylaryl or alkoxyaryl residue.
